(19) United States
(12) Patent Application Publication
(10) Pub. No.: US 2008/0207665 A1
(43) Pub. Date: Aug. 28, 2008

(54) Alpha-(Aryl-or Heteroaryl-Methyl)-Beta-Piperidinopropanoic Acid Compounds as Orl-1-Receptor Antagonists

(75) Inventors: Yoshinobu Hashizume, Aichi-ken (JP); Masako Hirota, Aichi-ken (JP); Sachiko Mihara, Aichi-ken (JP); Hiroshi Nakamura, Aichi-ken (JP); Hiroki Koike, Aichi-ken (JP); Yukari Matsumoto, Aichi-ken (JP)

Correspondence Address: PFIZER INC., PATENT DEPARTMENT, MS8260-1611, EASTERN POINT ROAD, GROTON, CT, 06340, US

(73) Assignee: Pfizer Inc.

(21) Appl. No.: 11/917,210

(22) PCT Filed: Jun. 8, 2006
(86) PCT No.: PCT/IB06/01624

§ 371 Date: Apr. 18, 2008

Related U.S. Application Data
(60) Provisional application No. 60/691,905, filed on Jun. 17, 2005.

(51) Int. Cl. A61K 31/46; C07D 451/00

(52) U.S. Cl. 514/278; 546/18

(57) ABSTRACT

This invention provides the compounds of formula (I): or a pharmaceutically acceptable ester or salt thereof, wherein R¹ and R² independently represent hydrogen or the like; R³ represents aryl or the like; —X—Y— represents —CH₂O— or the like, and n represents 0, 1 or 2. These compounds have ORL1-receptor antagonist activity; and therefore, are useful to treat diseases or conditions such as pain, various CNS diseases etc.

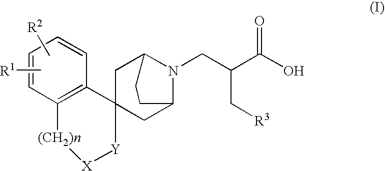

### TECHNICAL FIELD

This invention relates to alpha-(aryl- or heteroaryl-methyl)-beta-piperidinopropanoic acid compounds, and pharmaceutically acceptable esters or salts thereof, and to medical uses thereof. Also, this invention relates to pharmaceutical compositions comprising said compounds, or their pharmaceutically acceptable ester or salt. The compounds of this invention have binding affinity for the ORL-1 receptor. In particular, the compounds of this invention have antagonist activity for said receptor. The compounds of this invention are useful in treating or preventing disorders or medical conditions selected from pain, a CNS disorder and the like, which are mediated by overactivation of said receptor.

### BACKGROUND ART

Three types of opioid receptors, μ (mu), δ (delta) and κ (kappa) have been identified. These receptors may be indicated with combinations of OP (abbreviation for Opioid Peptides) and numeric subscripts as suggested by the International Union of Pharmacology (IUPHAR). Namely, OP₁, OP₂ and OP₃ respectively correspond to δ-, κ- and μ-receptors. They are known to belong to the G-protein-coupled receptors and are distributed in the central nervous system (CNS), peripheries and organs in a mammal. Endogenous and synthetic opioids are known as ligands for the receptors. It is believed that an endogenous opioid peptide produces its effects through an interaction with the major classes of opioid receptors. For example, endorphins have been purified as endogenous opioid peptides and bind to both δ- and μ-receptors. Morphine is a well-known non-peptide opioid analgesic and has binding affinity mainly for the Preceptor. Opiates have been widely used as pharmacological agents, but drugs such as morphine and heroin induce some side effects such as drug addiction and euphoria.

Meunier et al. reported isolation of a seventeen-amino-acid-long peptide from rat brain as an endogenous ligand for an orphan opioid receptor (Nature, Vol. 337, pp. 532-535, Oct. 12, 1995), and said receptor is now known as the “opioid receptor-like 1 (abbreviated as ORL-1) receptor”. In the same report, the endogenous opioid ligand was disclosed as an agonist for the ORL-1 receptor and named as “nociceptine (abbreviated as NC)”. Also, the same ligand was named as “orphanin FQ (abbreviated as OFQ or oFQ)” by Reinscheid et al. (Science, Vol. 270, pp. 792-794, 1995). This receptor may also be indicated as OP₄ in line with a recommendation by IUPHAR in 1998 (British Journal of Pharmacology, Vol. 129, pp. 1261-1283, 2000).

International Patent Application Number (WO) 9429309 discloses a variety of spiro-substituted azacycle compounds, which are Neurokinin antagonists useful in the treatment of pain.

Also, International Patent Application Number (WO) 9825605 discloses a variety of spiro-substituted azacycle compounds, which are Chemokine receptor activity modulator antagonists.

Further, International Patent Application Number (WO) 0226714 discloses a variety of spiropiperidino compounds which show a binding affinity to a Nociceptin receptor.

Yet further, International Patent Application Number (WO) 03064425 discloses a variety of spiropiperidino compounds, which are ORL1 antagonists, for example, compound (i) below:

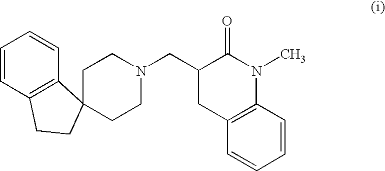

Compound (i) shows a potent activity in the dofetilide binding assay and thus high predicted HERG potassium channel inhibitory activity.

There is a need to provide new ORL1 antagonists that are good drug candidates and which potentially have improved properties (e.g. greater potency, greater selectivity, better absorption from the gastrointestinal tract, greater metabolic stability and more favourable pharmacokinetic properties). Other potential advantages include greater or lesser penetration of the blood brain barrier, according to the disease targeted, lower toxicity and a decreased incidence of side-effects. In particular, preferred compounds should bind potently to the ORL1 receptor and show functional activity as antagonists whilst showing little affinity for other receptors. Furthermore, it would be desirable to provide an ORL1 antagonist with reduced inhibitory activity at the HERG potassium channel.

### BRIEF DISCLOSURE OF THE INVENTION

It has now surprisingly been found that the alpha aryl or heteroaryl methyl beta piperidino propanoic acid compounds of the present invention are ORL1 antagonists with analgesic activity, particularly when given by systemic administration, and reduced inhibitory activity on the HERG channel. Preferred compounds of the present invention also showed a reduced QT prolongation.

The present invention provides a compound of the following formula (I):

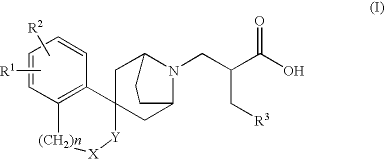

or a pharmaceutically acceptable ester or salt thereof,
wherein R¹ and R² independently represent hydrogen, halogen or (C₁-C₃)alkyl; R³ represents aryl or heteroaryl, each optionally substituted by 1 to 3 substituents independently selected from halogen, hydroxy, (C₁-C₃)alkyl or (C₁-C₃)alkoxy, heteroaryl is a 5- or 6-membered aromatic heterocyclic group comprising either (a) 1 to 4 nitrogen atoms, (b) one oxygen or one sulphur atom or (c) 1 oxygen atom or 1 sulphur atom and 1 or 2 nitrogen atoms; —X—Y— represents —CH₂O—, —CH(CH₃)O— or C(CH₃)₂O—; and n represents 0, 1 or 2.

The compounds of the present invention are antagonists of the ORL1 receptor, and have a number of therapeutic applications, particularly in the treatment of pain including inflammatory pain and neuropathic pain.

The compounds of the present invention are useful for the general treatment of pain.

Pain may generally be classified as acute or chronic. Acute pain begins suddenly and is short-lived (usually in twelve weeks or less). It is usually associated with a specific cause such as a specific injury and is often sharp and severe. It is the kind of pain that can occur after specific injuries resulting from surgery, dental work, a strain or a sprain. Acute pain does not generally result in any persistent psychological response. In contrast, chronic pain is long-term pain, typically persisting for more than three months and leading to significant psychological and emotional problems. Common examples of chronic pain are neuropathic pain (e.g. painful diabetic neuropathy, postherpetic neuralgia), carpal tunnel syndrome, back pain, headache, cancer pain, arthritic pain and chronic post-surgical pain.

When a substantial injury occurs to body tissue, via disease or trauma, the characteristics of nociceptor activation are altered and there is sensitisation in the periphery, locally around the injury and centrally where the nociceptors terminate. These effects lead to a hightened sensation of pain. In acute pain these mechanisms can be useful, in promoting protective behaviours which may better enable repair processes to take place. The normal expectation would be that sensitivity returns to normal once the injury has healed. However, in many chronic pain states, the hypersensitivity far outlasts the healing process and is often due to nervous system injury. This injury often leads to abnormalities in sensory nerve fibres associated with maladaptation and aberrant activity (Woolf & Salter, 2000, Science, 288, 1765-1768).

Clinical pain is present when discomfort and abnormal sensitivity feature among the patient's symptoms. Patients tend to be quite heterogeneous and may present with various pain symptoms. Such symptoms include: 1) spontaneous pain which may be dull, burning, or stabbing; 2) exaggerated pain responses to noxious stimuli (hyperalgesia); and 3) pain produced by normally innocuous stimuli (allodynia—Meyer et al., 1994, Textbook of Pain, 13-44). Although patients suffering from various forms of acute and chronic pain may have similar symptoms, the underlying mechanisms may be different and may, therefore, require different treatment strategies. Pain can also therefore be divided into a number of different subtypes according to differing pathophysiology, including nociceptive, inflammatory and neuropathic pain.

Neuropathic pain is currently defined as pain initiated or caused by a primary lesion or dysfunction in the nervous system. Nerve damage can be caused by trauma and disease and thus the term ‘neuropathic pain’ encompasses many disorders with diverse aetiologies. These include, but are not limited to, peripheral neuropathy, diabetic neuropathy, post herpetic neuralgia, trigeminal neuralgia, back pain, cancer neuropathy, HIV neuropathy, phantom limb pain, carpal tunnel syndrome, central post-stroke pain and pain associated with chronic alcoholism, hypothyroidism, uremia, multiple sclerosis, spinal cord injury, Parkinson's disease, epilepsy and vitamin deficiency.

The inflammatory process is a complex series of biochemical and cellular events, activated in response to tissue injury or the presence of foreign substances, which results in swelling and pain (Levine and Taiwo, 1994, Textbook of Pain, 45-56). Arthritic pain is the most common inflammatory pain. Rheumatoid disease is one of the commonest chronic inflammatory conditions in developed countries and rheumatoid arthritis is a common cause of disability.

Another type of inflammatory pain is visceral pain which includes pain associated with inflammatory bowel disease (IBD). Visceral pain is pain associated with the viscera, which encompass the organs of the abdominal cavity. These organs include the sex organs, spleen and part of the digestive system. Pain associated with the viscera can be divided into digestive visceral pain and non-digestive visceral pain. Commonly encountered gastrointestinal (GI) disorders that cause pain include functional bowel disorder (FBD) and inflammatory bowel disease (IBD). These GI disorders include a wide range of disease states that are currently only moderately controlled, including, in respect of FBD, gastro-esophageal reflux, dyspepsia, irritable bowel syndrome (IBS) and functional abdominal pain syndrome (FAPS), and, in respect of IBD, Crohn's disease, ileitis and ulcerative colitis, all of which regularly produce visceral pain. Other types of visceral pain include the pain associated with dysmenorrhea, cystitis and pancreatitis and pelvic pain.

Apart from pain, the compounds of formula (I) are also potentially useful in the treatment of any disease or condition which is treatable using an ORL-1 antagonist. Such conditions include sleep disorders, eating disorders including anorexia and bulimia; anxiety and stress conditions; immune system diseases; locomotor disorder; memory loss, cognitive disorders and dementia including senile dementia, Alzheimer's disease, Parkinsons disease or other neurodegenerative pathologies; epilepsy or convulsion and symptoms associated therewith; a central nervous system disorder related to glutamate release action, anti-epileptic action, disruption of spatial memory, serotonin release, anxiolytic action, mesolimbic dopaminergic transmission, rewarding propaerties of drug of abuse, modulation of striatal and glutamate effects on locomotor activity; cardiovascular disorders including hypotension, bradycardia and stroke; renal disorders including water excretion, sodium ion excretion and syndrome of inappropriate secretion of antidiuretic hormone (SIADH); gastrointestinal disorders; airway disorders including adult respiratory distress syndrome (ARDS); metabolic disorders including obesity; cirrhosis with ascites; sexual dysfunctions; altered pulmonary function including obstructive pulmonary disease, and tolerance to or dependency on a narcotic analgesic or the like.

Thus, the present invention relates to a compound of the formula (I) for use as a medicament.

As a yet further aspect of the present invention, there is provided the use of a compound of formula (I), or a pharmaceutically acceptable ester or salt thereof, in the manufacture of a medicament for the treatment of pain.

As an alternative aspect, there is provided a method for the treatment of pain comprising administration of a therapeutically effective amount of a compound of formula (I), or a pharmaceutically acceptable ester or salt thereof, to a mammal in need of said treatment.

### DETAILED DESCRIPTION OF THE INVENTION

As used herein, the term “halogen” means fluoro, chloro, bromo or iodo, preferably fluoro or chloro.

As used herein, the term “(C₁-C₃)alkyl” means a straight or branched chain saturated monovalent hydrocarbon radical, including, but not limited to methyl, ethyl, n-propyl and isopropyl.

As used herein, the term “(C₁-C₃)alkoxy” means alkyl-O-, including, but not limited to methoxy, ethoxy, n-propoxy, isopropoxy.

As used herein, the term “aryl” means phenyl or naphthyl, preferably phenyl.

As used herein, the term “heteroaryl” means a 5- or 6-membered aromatic heterocyclic group comprising either (a) 1 to 4 nitrogen atoms, (b) one oxygen or one sulphur atom or (c) 1 oxygen atom or 1 sulphur atom and 1 or 2 nitrogen atoms including, but not limited to, pyrazolyl, furyl, thienyl, oxazolyl, tetrazolyl, thiazolyl, imidazolyl, thiadiazolyl, pyridyl, pyrimidinyl, pyrrolyl, thiophenyl, pyrazinyl, pyridazinyl, isooxazolyl, isothiazolyl, triazolyl, furazanyl, quinolyl, isoquinolyl, tetrahydroquinolyl, tetrahydroisoquinolyl, chromanyl or isochromanyl, and the like.

The term “protecting group” means a group, which can be cleaved by a chemical method such as hydrogenolysis, hydrolysis, electrolysis or photolysis. Where the compounds of formula (I) contain hydroxy groups, they may form esters. Examples of such esters include esters with a hydroxy group and esters with a carboxy group. The ester residue may be an ordinary protecting group or a protecting group which can be cleaved in vivo by a biological method such as hydrolysis.

In a preferred aspect (A), the invention provides a compound of the formula (I), or a pharmaceutically acceptable ester or salt thereof, wherein R¹ and R² independently represent hydrogen or halogen; more preferably hydrogen or fluorine; most preferably R¹ and R² represent hydrogen, or R¹ represents hydrogen and R² represents fluorine; and R³, X, Y and n are as defined above.

In a further preferred aspect (B), the invention provides a compound of the formula (I), or a pharmaceutically acceptable ester or salt thereof, wherein R¹ and R² are defined above, either in the broadest aspect or in a preferred, more or most preferred aspect under (A); R³ represents phenyl or heteroaryl wherein heteroaryl is a 5- to 6-membered heteroaromatic group containing from 1 to 2 nitrogen heteroatoms or 1 or 2 nitrogen heteroatoms and 1 oxygen or 1 sulfur atom, and said phenyl and heteroaryl are optionally substituted by 1 to 2 substituents each independently selected from halogen or hydroxy; more preferably, R³ represents phenyl, thiazolyl, isothiazolyl, pyrazolyl, imidazolyl, isoxazolyl or oxazolyl, each optionally substituted by 1 to 2 substituents each independently selected from chlorine or hydroxy; most preferably, R³ represents phenyl, thiazol-4-yl, or pyrazol-1-yl, each optionally substituted by 1 to 2 substituents each independently selected from chlorine or hydroxyl; and X, Y and n are as defined above.

In a further preferred aspect (C), the invention provides a compound of the formula (I), or a pharmaceutically acceptable ester or salt thereof, wherein R¹, R² and R³ are defined above, either in the broadest aspect or in a preferred, more or most preferred aspect under (A) or (B); —X—Y— represents —CH₂O— and n represents 0 or 1.

Individual preferred R¹ through R³ and X, Y and n groups are those defined by the R¹ through R³ and X, Y and n groups in the Examples section below.

Particularly preferred compounds of the invention include those in which each variable in Formula (I) is selected from the preferred groups for each variable. Even more preferable compounds of the invention include those where each variable in Formula (I) is selected from the more or most preferred groups for each variable.

A specific preferred compound according to the invention is selected from the list consisting of:
3-(3′H,8H-Spiro[8-azabicyclo[3.2.1]octane-3,1′-[2]benzofuran]-8-yl)-2-(1,3-thiazol-4-ylmethyl)propanoic acid;
3-(1H-Pyrazol-1-yl)-2-(3′H,8H-spiro[8-azabicyclo[3.2.1]octane-3,1′-[2]benzofuran]-8-ylmethyl)propanoic acid;
6′-fluoro-3′H, 8H-spiro[8-azabicyclo[3.2.1]octane-3,1′-[2]benzofuran]-8-carboxylate;
3-(6′-Fluoro-3′H,8H-spiro[8-azabicyclo[3.2.1]octane-3,1′-[2]benzofuran]-8-yl)-2-(1,3-thiazol-4-ylmethyl)propanoic acid;
3-(3′,4′-Dihydro-8H-spiro[8-azabicyclo[3.2.1]octane-3,1′-isochromen]-8-yl)-2-(1H-pyrazol-1-ylmethyl)propanoic acid;
3-(6′-Fluoro-3′,4′-dihydro-8H-spiro[8-azabicyclo[3.2.1]octane-3,1′-isochromen]-8-yl)-2-(1H-pyrazol-1-8-ylmethyl)propanoic acid;
2-(2-Chlorobenzyl)-3-(6′-fluoro-3′,4′-dihydro-8H-spiro[-azabicyclo[3.2.1]octane-3,1′-isochromen]-8-yl)propanoic acid;
2-(2-Chlorobenzyl)-3-(6′-fluoro-3′H,8H-spiro[8-azabicyclo[3.2.1]octane-3,1′-[2]benzofuran]-8-yl)propanoic acid;
2-(2-Chloro-5-hydroxybenzyl)-3-(6′-fluoro-3′,4′-dihydro-8H-spiro[8-azabicyclo[3.2.1]octane-3,1′-isochromen]-8-yl)propanoic acid; and
2-(2-Chloro-5-hydroxybenzyl)-3-(6′-fluoro-3′H,8H-spiro[8-azabicyclo[3.2.1]octane-3,1′-[2]benzofuran]-8-yl)propanoic acid;
and the pharmaceutically acceptable esters and salts thereof.

### General Synthesis:

The compounds of formula I of the present invention may be prepared according to known preparation methods, or the general procedures or preparation methods illustrated in the following reaction schemes. Unless otherwise indicated, R¹ through R³ and X, Y, and n in the reaction schemes and discussion that follow are defined as above. The term “protecting group”, as used hereinafter, means a hydroxy or amino protecting group which is selected from typical hydroxy or amino protecting groups described in Protective Groups in Organic Synthesis edited by T. W. Greene et al. (John Wiley & Sons, 1999);

According to a first process, the compounds of formula (I) may be prepared from compounds of formula 1-11 as illustrated in Scheme 1.

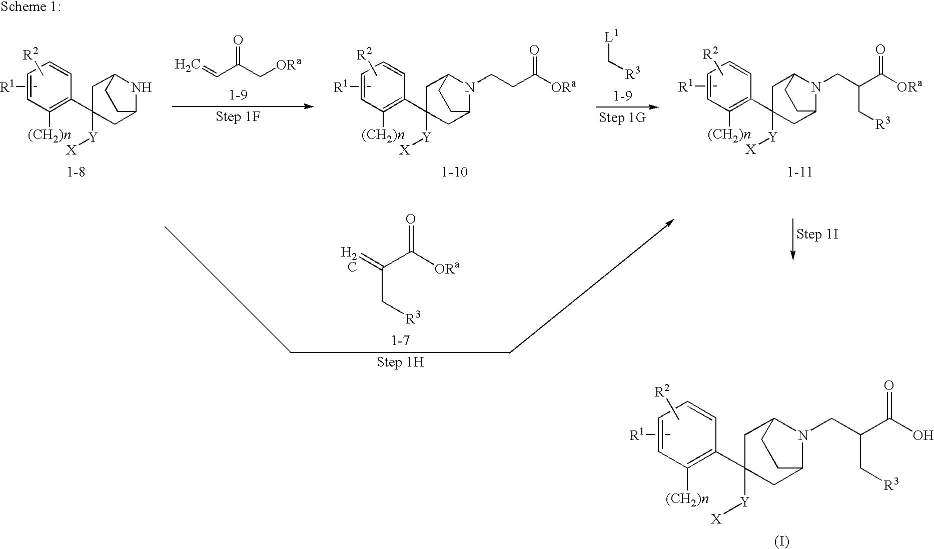

wherein R^{a} represents (C₁-C₄)alkyl;. L¹ represents a suitable leaving group, for example halogen atoms, such as chlorine, bromine and iodine; sulfonic esters such as TfO (triflates), MsO (mesylates), TsO (tosylates); and the like.

### Step 1F

In this step, the compounds of formula 1-8 can be prepared according to literature methods (Bioorg. Med. Chem. Lett. 1998, 8, 1541.). A compound of formula 1-10 can be prepared by Michael reaction of a compound of formula 1-8 with an enone compound of formula 1-9 in the presence of a base in a reaction-inert solvent. Examples of suitable solvents include: acetonitrile, tetrahydrofuran, N,N-dimethylformamide, dimethylsulfoxide, ether, toluene, ethylene glycol dimethylether, water and 1,4-dioxane. Examples of suitable bases include: triethylamine, tributylamine, diisopropylethylamine, pyridine, picoline, N-methylmorpholine and N-methylpiperidine, sodium carbonate, potassium carbonate, sodium bicarbonate, cesium carbonate. This reaction may be carried out at a temperature in the range from 0° C. to 200° C., usually from 25° C. to 100° C., for from 5 minutes to 60 hours, usually from 30 minutes to 30 hours.

### Step 1G

In this step, a compound of formula 1-11 can be prepared by alkylation of a compound of formula 1-10 with an alkylating agent of the formula 1-2 in the presence of a base in a reaction-inert solvent. Examples of suitable solvents include: tetrahydrofuran, diethylether, toluene, ethylene glycol dimethylether and 1,4-dioxane. Examples of suitable bases include: lithium bis(trimethylsilyl)amide; sodium bis(trimethylsilyl)amide; potassium bis(trimethylsilyl)amide; metal amide such as sodium amide or lithium diisopropylamide; and alkali metal hydride, such as potassium hydride or sodium hydride. If desired, this reaction may be carried out in the presence or absence of an additive such as N,N′-dimethylpropyleneurea (DMPU), hexamethylphosphoramide (HMPA), or N,N,N′,N′-tetramethylethylenediamine (TMEDA). This reaction may be carried out at a temperature in the range from −100° C. to 200° C., usually from −80° C. to 100° C., for from 5 minutes to 72 hours, usually from 30 minutes to 36 hours.

### Step 1H

Alternatively, a compound of formula 1-11 can be prepared directly from a compound of formula 1-8 by Michael reaction with an enone compound of formula 1-7 in the presence or absence of a base in a reaction-inert solvent. Examples of suitable solvents include: methanol, ethanol, tetrahydrofuran, N,N-dimethylformamide, dimethylsulfoxide, diethylether, toluene, ethylene glycol dimethylether, water and 1,4-dioxane. Examples of suitable bases include: triethylamine, tributylamine, diisopropylethylamine, pyridine, picoline, N-methylmorpholine and N-methylpiperidine. This reaction may be carried out at a temperature in the range from 0° C. to 200° C., usually from 25° C. to 100° C., for from 1 hour to 2 weeks, usually from 5 hours to 10 days.

### Step 1l

In this step, an acid compound of formula (I) may be prepared by hydrolysis of an ester compound of formula 1-11 in a solvent. The hydrolysis may be carried out by conventional procedures. In a typical procedure, the hydrolysis is carried out under basic conditions, e.g. in the presence of sodium hydroxide, potassium hydroxide or lithium hydroxide. Suitable solvents include, for example, alcohols such as methanol, ethanol, propanol, butanol, 2-methoxyethanol, and ethylene glycol; ethers such as tetrahydrofuran (THF), 1,2-dimethoxyethane (DME), and 1,4-dioxane; amides such as N,N-dimethylformamide (DMF) and hexamethylphospholictriamide; and sulfoxides such as dimethyl sulfoxide (DMSO). This reaction may be carried out at a temperature in the range from −20° C. to 100° C., usually from 20° C. to 75° C., for from 30 minutes to 48 hours, usually from 60 minutes to 30 hours.

The hydrolysis may alternatively be carried out under acidic conditions, e.g. in the presence of hydrogen halides, such as hydrogen chloride and hydrogen bromide; sulfonic acids, such as p-toluenesulfonic acid and benzenesulfonic acid; pyridium p-toluenesulfonate; or carboxylic acids, such as acetic acid and trifluoroacetic acid. Suitable solvents include, for example, alcohols such as methanol, ethanol, propanol, butanol, 2-methoxyethanol, and ethylene glycol; ethers such as tetrahydrofuran (THF), 1,2-dimethoxyethane (DME), and 1,4-dioxane; halogenated hydrocarbons, such as dichloromethane, 1,2-dichloroethane; amides such as N,N-dimethylformamide (DMF) and hexamethylphospholictriamide; and sulfoxides such as dimethyl sulfoxide (DMSO). This reaction may be carried out at a temperature in the range from −20° C. to 100° C., usually from 0° C. to 65° C., for from 30 minutes to 24 hours, usually from 60 minutes to 10 hours.

Compounds of formula 1-7 may be prepared from compounds of formula 1-4 as illustrated in Scheme 1.1

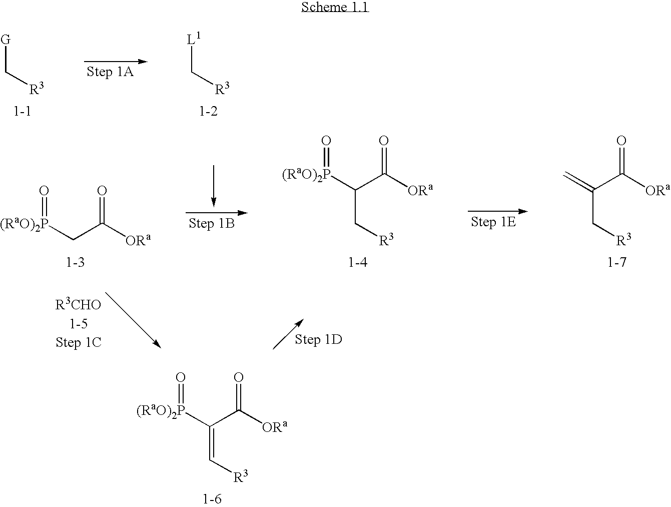

wherein G represents hydrogen or hydroxy and L¹ and R^{a} are as defined above for Scheme 1.

### Step 1A

In this step, when L¹ represents halogen, a compound of the formula 1-2 can be prepared by halogenating a compound of the formula 1-1 in which G represents a hydrogen atom under halogenation conditions with a halogenating reagent in a reaction-inert solvent. When R³ is substituted by a hydroxy group, the hydroxy group is protected with a protecting group according to conventional methods.

Examples of suitable solvents include: tetrahydrofuran; 1,4-dioxane; N,N-dimethylformamide; acetonitrile; alcohols, such as methanol or ethanol; halogenated hydrocarbons, such as dichloromethane, 1,2-dichloroethane, chloroform or carbon tetrachloride; and acetic acid. Suitable halogenating reagents include, for example, bromine, chlorine, iodine, N-chlorosuccinimide, N-bromosuccinimide, 1,3-dibromo-5,5-dimethylhydantoin, bis(dimethylacetamide)hydrogen tribromide, tetrabutylammonium tribromide, bromodimethylsulfonium bromide, hydrogen bromide-hydrogen peroxide, nitrodibromoacetonitrile or copper(II) bromide. The reaction can be carried out at a temperature of from 0° C. to 200° C., more preferably from 20° C. to 120° C. Reaction times are, in general, from 5 minutes to 48 hours, more preferably 30 minutes to 24 hours.

When L¹ represents a halogen atom or a sulfonic ester, a compound of the formula 1-2 can be prepared by halogenating or sulfonating a compound of the formula 1-1 in which G represents a hydroxy group under conditions known to those skilled in the art.

For example, the hydroxy group of the compound of formula 1-1 may be replaced with a halogen atom using a halogenating agent in the presence or absence of a reaction inert solvent. Preferred halogenating agents include: chlorinating agents, such as thionyl chloride, oxalyl chloride, p-toluenesulfonyl chloride, methanesulfonyl chloride, hydrogen chloride, phosphorus trichloride, phosphorus pentachloride or phosphorus oxychloride; and phosphorus reagents such as triphenylphosphine, tributyl phosphine or triphenylphosphite in the presence of a halogen source such as carbon tetrachloride, chlorine, N-chlorosuccinimide (NCS), hydrogen bromide, N-bromosuccinimide (NBS), phosphorus tribromide, trimethylsilyl bromide, hydroiodic acid, phosphorus triiodide, or iodine. Examples of suitable solvents include: aliphatic hydrocarbons, such as hexane, heptane and petroleum ether; aromatic hydrocarbons, such as benzene, toluene, o-dichlorobenzene, nitrobenzene, pyridine, and xylene; halogenated hydrocarbons, such as dichloromethane, chloroform, carbon tetrachloride and 1,2-dichloroethane; and ethers, such as diethyl ether, diisopropyl ether, tetrahydrofuran and 1,4-dioxane. This reaction may be carried out at a temperature in the range from −100° C. to 250° C., more preferably from 0° C. to the reflux temperature, for 1 minute to a day, more preferably from 20 minutes to 5 hours.

Alternatively, the hydroxy group of the compound of formula 1-1 may be replaced with a sulfonate group using a sulfonating agent in the presence or absence of a base. Examples of such sulfonating agents include: p-toluenesulfonyl chloride, p-toluenesulfonic anhydride, methanesulfonyl chloride, methanesulfonic anhydride, trifluoromethanesulfonic anhydride, or the like, in the presence or absence of a reaction-inert solvent. Example of suitable bases include: an alkali or alkaline earth metal hydroxide, alkoxide, carbonate, halide or hydride, such as sodium hydroxide, potassium hydroxide, sodium methoxide, sodium ethoxide, potassium tert-butoxide, sodium carbonate, potassium carbonate, potassium fluoride, sodium hydride or potassium hydride; or an amine such as triethylamine, tributylamine, diisopropylethylamine, pyridine or dimethylaminopyridine, in the presence or absence of a reaction-inert solvent. Examples of suitable solvents include: aliphatic hydrocarbons, such as hexane, heptane and petroleum ether; aromatic hydrocarbons, such as benzene, toluene, o-dichlorobenzene, nitrobenzene, pyridine, and xylene; halogenated hydrocarbons, such as dichloromethane, chloroform, carbon tetrachloride and 1,2-dichloroethane; ethers, such as diethyl ether, diisopropyl ether, tetrahydrofuran and 1,4-dioxane; N,N-dimethylformamide; and dimethylsulfoxide. This reaction may be carried out at a temperature in the range from −50° C. to 100° C., more preferably from −10° C. to 50° C. for 1 minute to a day, more preferably from 20 minutes to 5 hours.

### Step 1B

In this step, a compound of formula 1-4 can be prepared by alkylation of a compound of formula 1-3 with an alkylating agent of formula 1-2 in the presence of a base in a reaction-inert solvent. Examples of suitable solvents include: tetrahydrofuran, N,N-dimethylformamide, dimethylsulfoxide, diethylether, toluene, ethylene glycol dimethylether and 1,4-dioxane. Examples of suitable bases include: alkyl lithiums, such as n-butyllithium, secbutyllithium or tert-butyllithium; aryllithiums, such as phenyllithium or lithium naphthylide; metal amides such as sodium amide or lithium diisopropylamide; and alkali metal hydrides such as potassium hydride or sodium hydride. This reaction may be carried out at a temperature in the range from −50° C. to 200° C., usually from −10° C. to 100° C. for 5 minutes to 72 hours, usually 30 minutes to 36 hours.

### Step 1C

In this step, a compound of formula 1-6 can be prepared by aldol condensation of a compound of formula 1-3 with an aldehyde compound of formula 1-5 in the presence of a base in a reaction-inert solvent. Examples of suitable solvents include: tetrahydrofuran, N,N-dimethylformamide, dimethylsulfoxide, ether, toluene, ethylene glycol dimethylether and 1,4-dioxane. Examples of suitable bases include: lithium hydroxide, sodium hydroxide, potassium hydroxide, barium hydroxide, sodium carbonate, potassium carbonate, sodium bicarbonate, cesium carbonate, thallium(l) carbonate, sodium ethoxide, potassium tert-butoxide, potassium acetate, cesium fluoride, tetrabutylammonium fluoride, tetrabutylammonium chloride, tetrabutylammonium iodide, pyridine, picoline, 4-(N,N-dimethylamino)pyridine, triethylamine, tributylamine, diisopropylethylamine, N-methylmorpholine and N-methylpiperidine. This reaction may be carried out at a temperature in the range from −50 ° C. to 250 ° C., usually from −10 ° C. to 150 ° C. for 5 minutes to 72 hours, usually 30 minutes to 24 hours.

### Step 1D

In this step, the compound of formula 1-4 can be prepared by reduction of an olefin compound of formula 1-6 with a reducing agent in an inert solvent. Examples of suitable solvents include: methanol, ethanol, ethyl acetate, tetrahydrofuran (THF) and mixtures thereof. The reduction may be carried out under known hydrogenation conditions in the presence of a metal catalyst, e.g. nickel catalysts such as Raney nickel, palladium catalysts such as Pd—C, platinum catalysts such as PtO₂, or ruthenium catalysts such as RuCl₂ (Ph₃P)₃, under a hydrogen atmosphere or in the presence of hydrogen sources such as hydrazine or formic acid. If desired, the reaction may be carried out under acidic conditions, e.g. in the presence of hydrochloric acid or acetic acid. This reaction may be carried out at a temperature in the range from −50° C. to 200° C., usually from −10° C. to 100° C., for 5 minutes to 72 hours, usually 30 minutes to 36 hours.

### Step 1E

In this step, a compound of formula 1-7 can be prepared by Horner-Emmons reaction of a compound of formula 1-4 with formaldehyde or paraformaldehyde in the presence of a base in a reaction-inert solvent. Examples of suitable solvents include: tetrahydrofuran, N,N-dimethylformamide, dimethylsulfoxide, diethylether, toluene, ethylene glycol dimethylether, water and 1,4-dioxane. Examples of suitable bases include: lithium hydroxide, sodium hydroxide, potassium hydroxide, barium hydroxide, sodium carbonate, potassium carbonate, sodium bicarbonate, cesium carbonate, thallium(I) carbonate, sodium methoxide, sodium ethoxide, potassium tert-butoxide, potassium hydride and sodium hydride. This reaction may be carried out at a temperature in the range from 0° C. to 200° C., usually from 50° C. to 150° C., for 5 minutes to 72 hours, usually 30 minutes to 50 hours.

Alternatively, according to a second process, compounds of formula (I) may be prepared from compounds of formula 2-4 as illustrated in Scheme 2.

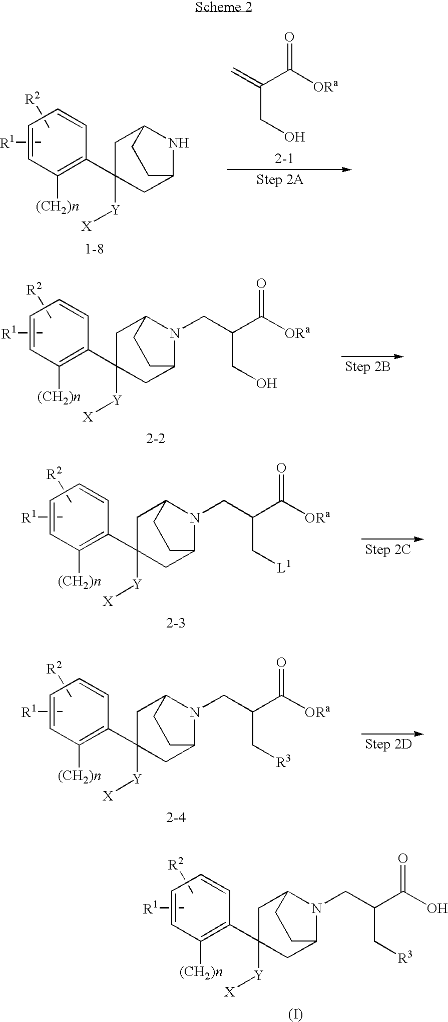

wherein, R^{a} and L¹ are as defined above for Scheme 1.

### Step 2A

In this step, a compound of formula 2-2 may be prepared by Michael reaction of a compound of formula 1-8 with an enone compound of formula 2-1. This reaction is essentially the same as and may be carried out in the same manner as and using the same reagents and reaction conditions as Step 1H in Scheme 1.

### Step 2B

In this step, a compound of formula 2-3 may be prepared from a compound of formula 2-2 under conditions known to those skilled in the art. This reaction is essentially the same as and may be carried out in the same manner as and using the same reagents and reaction conditions as Step 1A in Scheme 1.

### Step 2C

In this step, a compound of formula 2-4 can be prepared by reacting a compound of formula 2-3 with a compound of formula R³H in the presence of a base in a reaction-inert solvent. Examples of suitable solvents include: acetonitrile, tetrahydrofuran, N,N-dimethylformamide, dimethylsulfoxide, ether, toluene, ethylene glycol dimethylether and 1,4-dioxane. Examples of suitable bases include: lithium hydroxide, sodium hydroxide, potassium hydroxide, barium hydroxide, sodium carbonate, potassium carbonate, sodium bicarbonate, cesium carbonate, thallium(l) carbonate, sodium ethoxide, potassium tert-butoxide, potassium acetate, cesium fluoride, tetrabutylammonium fluoride, tetrabutylammonium chloride, tetrabutylammonium iodide, pyridine, picoline, 4-(N,N-dimethylamino)pyridine, triethylamine, tributylamine, diisopropylethylamine, N-methylmorpholine and N-methylpiperidine. This reaction may be carried out at a temperature in the range from 0° C. to 250° C., usually from −10° C. to 150° C., for 5 minutes to 72 hours, usually 30 minutes to 36 hours.

### Step 2D

In this step, a compound of formula (I) may be prepared by hydrolysis of a compound of formula 2-4. This reaction is essentially the same as and may be carried out in the same manner as and using the same reagents and reaction conditions as Step 1l in Scheme 1.

In the above Schemes, examples of suitable solvents include a mixture of any two or more of those solvents described in each step.

The starting materials in the aforementioned general syntheses are commercially available or may be obtained by conventional methods known to those skilled in the art.

The compounds of formula (I), and the intermediates in the above-mentioned preparation methods can be isolated and purified by conventional procedures, such as recrystallization or chromatographic purification.

The various general methods described above may be useful for the introduction of the desired groups at any stage in the stepwise formation of the required compound, and it will be appreciated that these general methods can be combined in different ways in such multi-stage processes. The sequence of the reactions in multi-stage processes should of course be chosen so that the reaction conditions used do not affect groups in the molecule which are desired in the final product.

### Method for Assessing Biological Activities:

The compounds of Formula (I) have been found to possess affinity for ORL1-receptors and ORL-1 receptor antagonist activity. Thus, these compounds are useful as an analgesic, anti-inflammatory, diuretic, anesthetic, neuroprotective, anti-hypertensive and anti-anxiety agent, and the like, in mammalian subjects, especially humans in need of such agents. The affinity, antagonist activities and analgesic activity can be demonstrated by the following tests respectively.

### Affinity for ORL1-Receptors:

### ORL1-Receptor Binding Assay:

The human ORL1 receptor transfected HEK-293 cell membranes (PerkinElmer) were incubated for 45 min at room temperature with 0.4 nM [³H]nociceptin, 1.0 mg of wheat germ agglutinin(WGA)-coated SPA beads and various concentrations of test compounds in a final volume of 200 μL of 50 mM HEPES buffer pH 7.4 containing 10 mM MgCl₂ and 1 mM EDTA. Non-specific binding (NSB) was determined by the addition of 1 μM unlabeled nociceptin. After the reaction, the assay plate was centrifuged at 1,000 rpm for 1 min and then the radioactivity was measured by WALLAC 1450 MicroBeta Trilux.

The compounds of the examples were tested in the ORL1 Receptor Binding assay. Ki values are presented in the following table.

| Example | Ki (nM) |
|---|---|
| 7 | 1.3 |
| 8 | 3.4 |
| 9 | 1.2 |
| 10 | 3.3 |

### μ-Receptor Binding Assay:

The human Mu receptor transfected CHO-K1 cell membranes (PerkinElmer) were incubated for 45 min at room temperature with 1.0 nM[³H]DAMGO, 1.0 mg of WGA-coated SPA beads and various concentrations of test compounds in a final volume of 200 μl of 50 mM Tris-HCl buffer pH 7.4 containing 5 mM MgCl₂. NSB was determined by the addition of 1 μM unlabeled DAMGO. After the reaction, the assay plate was centrifuged at 1,000 rpm for 1 min and then the radioactivity was measured by WALLAC 1450 MicroBata Trilux.

Each percent NSB thus obtained was graphed as a function of compound concentration. A sigmoidal curve was used to determine 50% bindings (i.e., IC₅₀ values).

In this testing, the preferred compounds prepared in the working examples appearing hereafter demonstrated higher binding affinity for ORL1-receptors than for mu-receptors.

IC₅₀ (ORL1-receptors) nM/IC₅₀ (mu-receptors) nM<1.0

### ORL1 Receptor Functional Assay:

The human ORL1 receptor transfected HEK-293 cell membranes were incubated with 400 pM [³⁵S]GTPγS, 10 nM nociceptin and various concentrations of test compounds in assay buffer (20 mM HEPES, 100 mM NaCl, 5 mM MgCl₂, 1 mM EDTA, 5 μM GDP, 1 mM DTT, pH 7.4) containing 1.5 mg of WGA-coated SPA beads for 90 min at room temperature in a final volume of 200 μL. Basal binding was assessed in the absence of nociceptin and NSB was defined by the addition of unlabelled 10 RM GTPγS. Membrane-bound radioactivity was detected by a Wallac 1450 MicroBeta liquid scintillation counter.

### Analgesic Tests:

### Tail Flick Test in Mice:

The latency time to withdrawal of the tail from radiant heat stimulation is recorded before and after administration of test compounds. Cut-off time is set to 8 sec.

### Acetic Acid Writhing Test in Mice:

Acetic acid saline solution of 0.7% (v/v) is injected intraperitoneally (0.16 mL/10 g body weight) to mice. Test compounds are administered before acetic acid injection. Immediately following acetic acid injection, the animals are placed in a 1 L beaker and writhing is recorded for 15 min.

### Formalin Licking Test in Mice:

Formalin-induced hind paw licking is initiated by a 20 μL subcutaneous injection of a 2% formalin solution into a hind paw of mice. Test compounds are administered prior to formalin injection. Total licking time is recorded for 45 min after formalin injection.

### Carrageenan-Induced Mechanical Hyperalgesia Test in Rats:

The response to mechanical nociceptive stimulus is measured using an algesiometer (Ugo Basile, Italy). The pressure is loaded to the paw until rats withdrawal the hind paw. Lambda-Carrageenan saline solution of 1% (w/v) is injected subcutaneously into the hind paw and the withdrawal response is measured before and after the injection. Test compounds are administered at an appropriate time point.

### Carrageenan-Induced Thermal Hyperalgesia Test in Rats:

The response to thermal nociceptive stimulus is measured using a plantar test apparatus (Ugo Basile, Italy). The radiant heat stimuli is applied to the paw until rats withdrawal the hind paw. Lambda-Carrageenan saline solution of 2% (w/v) is injected subcutaneously into the hind paw and the withdrawal response is measured before and after the injection. This testing method is described in K. Hargreaves, et al., Pain 32:77-88, 1988.

### Chronic Constriction Injury Model (CCI Model):

Chronic constriction injury is infilicted according to Bennett's method (Bennett and Xie, Pain 33:87-107, 1988). Tactile allodynia in rats is assessed using the von Frey hairs test (Stoelting, Ill.) before and after administration with test compounds.

### Partial Sciatic Nerve Ligation Model (PSL):

This test may be conducted according to similar procedures described by Z. Seltzer, et al. (A novel behavioral model of neuropathic pain disorders produced in rats by partial sciatic nerve injury: Pain, 43:205-218, 1990).

### Caco-2 Permeability

Caco-2 permeability was measured according to the method described by Shiyin Yee (Pharmaceutical Research, 763 (1997)).

### Human Dofetilide Binding Assay

Cell paste of HEK-293 cells expressing the HERG product was suspended in 10-fold volume of 50 mM Tris buffer adjusted at pH 7.5 at 25° C. with 2 M HCl containing 1 mM MgCl₂, 10 mM KCl. The cells were homogenized using a Polytron homogenizer (at the maximum power for 20 seconds) and centrifuged at 48,000 g for 20 minutes at 4° C. The pellet was resuspended, homogenized and centrifuged once more in the same manner. The resultant supernatant was discarded and the final pellet was resuspended (10-fold volume of 50 mM Tris buffer) and homogenized at the maximum power for 20 seconds. The membrane homogenate was aliquoted and stored at −80° C. until use. An aliquot was used for protein concentration determination using a Protein Assay Rapid Kit and ARVO SX plate reader (Wallac). All the manipulation, stock solution and equipment were kept on ice at all time. For saturation assays, experiments were conducted in a total volume of 200 μl. Saturation was determined by incubating 20 μl of [³H]-dofetilide and 160 μl of membrane homogenates (20-30 μg protein per well) for 60 min at room temperature in the absence or presence of 10 μM dofetilide at final concentrations (20 μl) for total or nonspecific binding, respectively. All incubations were terminated by rapid vacuum filtration over polyetherimide (PEI) soaked glass fiber filter papers using Skatron cell harvester followed by two washes with 50 mM Tris buffer (pH 7.5 at 25° C.). Receptor-bound radioactivity was quantified by liquid scintillation counting using Packard LS counter.

For the competition assay, compounds were diluted in 96 well polypropylene plates as 4-point dilutions in semi-log format. All dilutions were performed in DMSO first and then transferred into 50 mM Tris buffer (pH 7.5 at 25° C.) containing 1 mM MgCl₂, 10 mM KCl so that the final DMSO concentration became equal to 1%. Compounds were dispensed in triplicate in assay plates (4 μl). Total binding and nonspecific binding wells were set up in 6 wells as vehicle and 10 μM dofetilide at final concentration, respectively. The radioligand was prepared at 5.6× final concentration and this solution was added to each well (36 μl). The assay was initiated by addition of YSi poly-L-lysine Scintillation Proximity Assay (SPA) beads (50 μl, 1 mg/well) and membranes (110 μl, 20 μg/well). Incubation was continued for 60 min at room temperature. Plates were incubated for a further 3 hours at room temperature for beads to settle. Receptor-bound radioactivity was quantified by counting Wallac MicroBeta plate counter.

### I_{HERG} Assay

HEK 293 cells which stably express the HERG potassium channel were used for electrophysiological studies. The methodology for stable transfection of this channel in HEK cells can be found in the literature (Z. Zhou et al., 1998, Biophysical Journal, 74, pp 230-241). Before the day of experimentation, the cells were harvested from culture flasks and plated onto glass coverslips in a standard Minimum Essential Medium (MEM) medium with 10% Fetal Calf Serum (FCS). The plated cells were stored in an incubator at 37° C. maintained in an atmosphere of 95%O₂/5%CO₂. Cells were studied between 15-28 hrs after harvest.

HERG currents were studied using standard patch clamp techniques in the whole-cell mode. During the experiment the cells were superfused with a standard external solution of the following composition (mM); NaCl, 130; KCl, 4; CaCl₂, 2; MgCl₂, 1; Glucose, 10; HEPES, 5; pH 7.4 with NaOH. Whole-cell recordings was made using a patch clamp amplifier and patch pipettes which have a resistance of 1-3 MOhm when filled with the standard internal solution of the following composition (mM); KCl, 130; MgATP, 5; MgCl₂, 1.0; HEPES, 10; EGTA 5, pH 7.2 with KOH. Only those cells with access resistances below 15MΩ and seal resistances >1GΩ was accepted for further experimentation. Series resistance compensation was applied up to a maximum of 80%. No leak subtraction was done. However, acceptable access resistance depended on the size of the recorded currents and the level of series resistance compensation that can safely be used. Following the achievement of whole cell configuration and sufficient time for cell dialysis with pipette solution (>5 min), a standard voltage protocol was applied to the cell to evoke membrane currents. The voltage protocol is as follows. The membrane was depolarized from a holding potential of −80 mV to +40 mV for 100 ms. This was followed by a descending voltage ramp (rate 0.5 mV msec⁻¹) back to the holding potential. The voltage protocol was applied to a cell continuously throughout the experiment every 4 seconds (0.25 Hz). The amplitude of the peak current elicited around −40 mV during the ramp was measured. Once stable evoked current responses were obtained in the external solution, vehicle (0.5% DMSO in the standard external solution) was applied for 10-20 min by a peristalic pump. Provided there were minimal changes in the amplitude of the evoked current response in the vehicle control condition, the test compound of either 0.3, 1, 3, 10 μM was applied for a 10 min period. The 10 min period included the time which supplying solution was passing through the tube from solution reservoir to the recording chamber via the pump. Exposing time of cells to the compound solution was more than 5 min after the drug concentration in the chamber well reached the attempting concentration. There was a subsequent wash period of a 10-20 min to assess reversibility. Finally, the cells were exposed to high dose of dofetilide (5 μM), a specific IKr blocker, to evaluate the insensitive endogenous current.

All experiments were performed at room temperature (23±1° C.). Evoked membrane currents were recorded on-line on a computer, filtered at 500-1 KHz (Bessel −3 dB) and sampled at 1-2 KHz using the patch clamp amplifier and a specific data analyzing software. Peak current amplitude, which occurred at around −40 mV, was measured off line on the computer.

### Drug-Drug Interaction Assay

This method essentially involves determining the percent inhibition of product formation from fluorescence probe at 3 μM of the test compound.

More specifically, the assay is carried out as follows. The compounds were pre-incubated with recombinant CYPs, 100 mM potassium phosphate buffer and fluorescence probe as substrate for 5 min. Reaction was started by adding a warmed NADPH generating system, which consist of 0.5 mM NADP (expect; for 2D6 0.03 mM), 10 mM MgCl₂, 6.2 mM DL-lsocitric acid and 0.5 U/ml Isocitric Dehydrogenase (ICD). The assay plate was incubated at 37° C. (expect; for 1A2 and 3A4 at 30° C.) and fluorescence readings were taken every minute over 20 to 30 min.

### Half-Life in Human Liver Microsomes (HLM)

Test compounds (1 μM) were incubated with 3.3 mM MgCl₂ and 0.78 mg/mL HLM (HL101) in 100 mM potassium phosphate buffer (pH 7.4) at 37° C. on the 96-deep well plate. The reaction mixture was split into two groups, a non-P450 and a P450 group. NADPH was only added to the reaction mixture of the P450 group. An aliquot of samples of P450 group was collected at 0, 10, 30, and 60 min time point, where 0 min time point indicated the time when NADPH was added into the reaction mixture of P450 group. An aliquot of samples of non-P450 group was collected at -10 and 65 min time point. Collected aliquots were extracted with acetonitrile solution containing an internal standard. The precipitated protein was spun down in centrifuge (2000 rpm, 15 min). The compound concentration in supernatant was measured by LC/MS/MS system.

Pharmaceutically acceptable salts of the compounds of formula (I) include the acid addition and base salts thereof.

Suitable acid addition salts are formed from acids which form non-toxic salts. Examples include the acetate, aspartate, benzoate, besylate, bicarbonate/carbonate, bisulphate/sulphate, borate, camsylate, citrate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulphate, naphthylate, 2-napsylate, nicotinate, nitrate, orotate, oxalate, palmitate, pamoate, phosphate/hydrogen phosphate/dihydrogen phosphate, saccharate, stearate, succinate, tartrate, tosylate and trifluoroacetate salts.

Suitable base salts are formed from bases which form non-toxic salts. Examples include the aluminum, arginine, benzathine, calcium, choline, diethylamine, diolamine, glycine, lysine, magnesium, meglumine, olamine, potassium, sodium, tromethamine and zinc salts.

For a review on suitable salts, see “Handbook of Pharmaceutical Salts: Properties, Selection, and Use” by Stahl and Wermuth (Wiley-VCH, Weinheim, Germany, 2002).

A pharmaceutically acceptable salt of a compound of formula (I) may be readily prepared by mixing together solutions of the compound of formula (I) and the desired acid or base, as appropriate. The salt may precipitate from solution and be collected by filtration or may be recoveredby evaporation of the solvent. The degree of ionisation in the salt may vary from completely ionised to almost non-ionised.

The compounds of the invention may exist in both unsolvated and solvated forms. The term ‘solvate’ is used herein to describe a molecular complex comprising the compound of the invention and one or more pharmaceutically acceptable solvent molecules, for example, ethanol. The term ‘hydrate’ is employed when said solvent is water.

Included within the scope of the invention are complexes such as clathrates, drug-host inclusion complexes wherein, in contrast to the aforementioned solvates, the drug and host are present in stoichiometric or non-stoichiometric amounts. Also included are complexes of the drug containing two or more organic and/or inorganic components which may be in stoichiometric or non-stoichiometric amounts. The resulting complexes may be ionised, partially ionised, or non-ionised. For a review of such complexes, see J Pharm Sci, 64 (8), 1269-1288 by Haleblian (August 1975).

Hereinafter all references to compounds of formula (I) include references to salts, solvates and complexes thereof and to solvates and complexes of salts thereof.

The compounds of the invention include compounds of formula (I) as hereinbefore defined, polymorphs, prodrugs, and isomers thereof (including optical, geometric and tautomeric isomers) as hereinafter defined and isotopically-labeled compounds of formula (I).

As stated, the invention includes all polymorphs of the compounds of formula (I) as hereinbefore defined.

Also within the scope of the invention are so-called ‘prodrugs’ of the compounds of formula (I). Thus certain derivatives of compounds of formula (I) which may have little or no pharmacological activity themselves can, when administered into or onto the body, be converted into compounds of formula (I) having the desired activity, for example, by hydrolytic cleavage. Such derivatives are referred to as ‘prodrugs’. Further information on the use of prodrugs may be found in ‘Pro-drugs as Novel Delivery Systems, Vol. 14, ACS Symposium Series (T Higuchi and W Stella) and ‘Bioreversible Carriers in Drug Design’, Pergamon Press, 1987 (ed. E B Roche, American Pharmaceutical Association).

Prodrugs in accordance with the invention can, for example, be produced by replacing appropriate functionalities present in the compounds of formula (I) with certain moieties known to those skilled in the art as ‘pro-moieties’ as described, for example, in “Design of Prodrugs” by H Bundgaard (Elsevier, 1985).

Some examples of prodrugs in accordance with the invention include:
(i) where the compound of formula (I) contains a carboxylic acid functionality (—COOH), an ester thereof, for example, replacement of the hydrogen with (C₁-C₈)alkyl;
   (ii) where the compound of formula (I) contains an alcohol functionality (—OH), an ether thereof, for example, replacement of the hydrogen with (C₁-C₆)alkanoyloxymethyl; and
   (iii) where the compound of formula (I) contains a primary or secondary amino functionality (—NH₂ or —NHR where R≠H), an amide thereof, for example, replacement of one or both hydrogens with (C₁-C₁₀)alkanoyl.

Further examples of replacement groups in accordance with the foregoing examples and examples of other prodrug types may be found in the aforementioned references.

Finally, certain compounds of formula (I) may themselves act as prodrugs of other compounds of formula (I).

The term “ester” means a protecting group which can be cleaved in vivo by a biological method such as hydrolysis and forms a free acid or salt thereof. Whether a compound is such a derivative or not can be determined by administering it by intravenous injection to an experimental animal, such as a rat or mouse, and then studying the body fluids of the animal to determine whether or not the compound or a pharmaceutically acceptable salt thereof can be detected.

Preferred examples of groups for forming an ester with a hydroxy group and for forming an amide with a amino group include: (1) aliphatic alkanoyl groups, for example: alkanoyl groups such as the formyl, acetyl, propionyl, butyryl, isobutyryl, pentanoyl, pivaloyl, valeryl, isovaleryl, octanoyl, nonanoyl, decanoyl, 3-methylnonanoyl, 8-methylnonanoyl, 3-ethyloctanoyl, 3,7-dimethyloctanoyl, undecanoyl, dodecanoyl, tridecanoyl, tetradecanoyl, pentadecanoyl, hexadecanoyl, 1-methylpentadecanoyl, 14-methylpentadecanoyl, 13,13-dimethyltetradecanoyl, heptadecanoyl, 15-methylhexadecanoyl, octadecanoyl, 1-methylheptadecanoyl, nonadecanoyl, icosanoyl and henicosanoyl groups; halogenated alkylcarbonyl groups such as the chloroacetyl, dichloroacetyl, trichloroacetyl, and trifluoroacetyl groups; alkoxyalkanoyl groups such as the methoxyacetyl group; and unsaturated alkanoyl groups such as the acryloyl, propioloyl, methacryloyl, crotonoyl, isocrotonoyl and (E)-2-methyl-2-butenoyl groups; (2) aromatic alkanoyl groups, for example: arylcarbonyl groups such as the benzoyl, α-naphthoyl and β-naphthoyl groups; halogenated arylcarbonyl groups such as the 2-bromobenzoyl and 4-chlorobenzoyol groups; alkylated arylcarbonyl groups such as the 2,4,6-trimethylbenzoyl and 4-toluoyl groups; alkoxylated arylcarbonyl groups such as the 4-anisoyl group; nitrated arylcarbonyl groups such as the 4-nitrobenzoyl and 2-nitrobenzoyl groups; alkoxycarbonylated arylcarbonyl groups such as the 2-(methoxycarbonyl)benzoyl group; and arylated arylcarbonyl groups such as the 4-phenylbenzoyl group; (3) alkoxycarbonyl groups, for example: alkoxycarbonyl groups such as the methoxycarbonyl, ethoxycarbonyl, propoxycarbonyl, butoxycarbonyl, sec-butoxycarbonyl, t-butoxycarbonyl and isobutoxycarbonyl groups; and halogen- or tri(alkyl)silyl-substituted alkoxycarbonyl groups such as the 2,2,2-trichioroethoxycarbonyl and 2-trimethylsilylethoxycarbonyl groups; tetrahydropyranyl or tetrahydrothiopyranyl groups such as: tetrahydropyran-2-yl, 3-bromotetrahydropyran-2-yl, 4-methoxytetrahydropyran-4-yl, tetrahydrothiopyran-2-yl, and 4-methoxytetrahydrothiopyran-4-yl groups; tetrahydrofuranyl or tetrahydrothiofuranyl groups such as: tetrahydrofuran-2-yl and tetrahydrothiofuran-2-yl groups; (5) silyl groups, for example: tri(alkyl)silyl groups such as the trimethylsilyl, triethylsilyl, isopropyidimethylsilyl, t-butyldimethylsilyl, methyldiisopropylsilyl, methyldi-t-butylsilyl and triisopropylsilyl groups; and silyl groups substituted by one or more aryl and alkyl groups such as the diphenylmethylsilyl, diphenylbutylsilyl, diphenylisopropylsilyl and phenyldiisopropylsilyl groups; (6) alkoxymethyl groups, for example: alkoxymethyl groups such as the methoxymethyl, 1,1-dimethyl-1-methoxymethyl, ethoxymethyl, propoxymethyl, isopropoxymethyl, butoxymethyl and t-butoxymethyl groups; alkoxylated alkoxymethyl groups such as the 2-methoxyethoxymethyl group; and halo(alkoxy)methyl groups such as the 2,2,2-trichloroethoxymethyl and bis(2-chloroethoxy)methyl groups; (7) substituted ethyl groups, for example: alkoxylated ethyl groups such as the 1-ethoxyethyl and 1-(isopropoxy)ethyl groups; and halogenated ethyl groups such as the 2,2,2-trichloroethyl group; (8) aralkyl groups, for example: alkyl groups substituted by from 1 to 3 aryl groups such as the benzyl, a-naphthylmethyl, β-naphthylmethyl, diphenylmethyl, triphenylmethyl, α-naphthyldiphenylmethyl and 9-anthrylmethyl groups; alkyl groups substituted by from 1 to 3 substituted aryl groups, where one or more of the aryl groups is substituted by one or more alkyl, alkoxy, nitro, halogen or cyano substituents such as the 4-methylbenzyl, 2,4,6-trimethylbenzyl, 3,4,5-trimethylbenzyl, 4-methoxybenzyl, 4-methoxyphenyldiphenylmethyl, 2-nitrobenzyl, 4-nitrobenzyl, 4-chlorobenzyl, 4-bromobenzyl and 4-cyanobenzyl groups; alkenyloxycarbonyl groups such as the vinyloxycarbonyl; aryloxycarbonyl groups such as phenoxycaronyl; and aralkyloxycarbonyl groups in which the aryl ring may be substituted by 1 or 2 alkoxy or nitro groups, such as benzyloxycarbonyl, 4-methoxybenzyloxycarbonyl, 3,4-dimethoxybenzyloxycarbonyl, 2-nitrobenzyloxycarbonyl and 4-nitrobenzyloxycarbonyl groups.

Included within the scope of the present invention are all stereoisomers, geometric isomers and tautomeric forms of the compounds of formula (I), including compounds exhibiting more than one type of isomerism, and mixtures of one or more thereof. Also included are acid addition or base salts wherein the counterion is optically active, for example, D-lactate or L-lysine, or racemic, for example, DL-tartrate or DL-arginine.

Cis/trans isomers may be separated by conventional techniques well known to those skilled in the art, for example, chromatography and fractional crystallisation.

Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC).

Alternatively, the racemate (or a racemic precursor) may be reacted with a suitable optically active compound, for example, an alcohol, or, in the case where the compound of formula (I) contains an acidic or basic moiety, an acid or base such as tartaric acid or 1-phenylethylamine. The resulting diastereomeric mixture may be separated by chromatography and/or fractional crystallization and one or both of the diastereoisomers converted to the corresponding pure enantiomer(s) by means well known to a skilled person.

Chiral compounds of the invention (and chiral precursors thereof) may be obtained in enantiomerically-enriched form using chromatography, typically HPLC, on an asymmetric resin with a mobile phase consisting of a hydrocarbon, typically heptane or hexane, containing from 0 to 50% isopropanol, typically from 2 to 20%, and from 0 to 5% of an alkylamine, typically 0.1% diethylamine. Concentration of the eluate affords the enriched mixture.

Stereoisomeric conglomerates may be separated by conventional techniques known to those skilled in the art—see, for example, “Stereochemistry of Organic Compounds” by E L Eliel (Wiley, New York, 1994).

Compounds of the invention intended for pharmaceutical use may be administered as crystalline or amorphous products. They may be obtained, for example, as solid plugs, powders, or films by methods such as precipitation, crystallization, freeze drying, or spray drying, or evaporative drying. Microwave or radio frequency drying may be used for this purpose.

An ORL1 antagonist may be usefully combined with another pharmacologically active compound, or with two or more other pharmacologically active compounds, particularly in the treatment of pain. For example, an ORL1 antagonist, particularly a compound of formula (I), or a pharmaceutically acceptable salt or solvate thereof, as defined above, may be administered simultaneously, sequentially or separately in combination with one or more agents selected from:
an opioid analgesic, e.g. morphine, heroin, hydromorphone, oxymorphone, levorphanol, levallorphan, methadone, meperidine, fentanyl, cocaine, codeine, dihydrocodeine, oxycodone, hydrocodone, propoxyphene, nalmefene, nalorphine, naloxone, naltrexone, buprenorphine, butorphanol, nalbuphine or pentazocine;
a nonsteroidal antiinflammatory drug (NSAID), e.g. aspirin, diclofenac, diflusinal, etodolac, fenbufen, fenoprofen, flufenisal, flurbiprofen, ibuprofen, indomethacin, ketoprofen, ketorolac, meclofenamic acid, mefenamic acid, meloxicam, nabumetone, naproxen, nimesulide, nitroflurbiprofen, olsalazine, oxaprozin, phenylbutazone, piroxicam, sulfasalazine, sulindac, tolmetin or zomepirac;
a barbiturate sedative, e.g. amobarbital, aprobarbital, butabarbital, butabital, mephobarbital, metharbital, methohexital, pentobarbital, phenobartital, secobarbital, talbutal, theamylal or thiopental;
a benzodiazepine having a sedative action, e.g. chlordiazepoxide, clorazepate, diazepam, flurazepam, lorazepam, oxazepam, temazepam or triazolam;
an H₁ antagonist having a sedative action, e.g. diphenhydramine, pyrilamine, promethazine, chlorpheniramine or chlorcyclizine;
a sedative such as glutethimide, meprobamate, methaqualone or dichloralphenazone;
a skeletal muscle relaxant, e.g. baclofen, carisoprodol, chlorzoxazone, cyclobenzaprine, methocarbamol or orphrenadine;
an NMDA receptor antagonist, e.g. dextromethorphan ((+)-3-hydroxy-N-methylmorphinan) or its metabolite dextrorphan ((+)-3-hydroxy-N-methylmorphinan), ketamine, memantine, pyrroloquinoline quinine, cis-4-(phosphonomethyl)-2-piperidinecarboxylic acid, budipine, EN-3231 (MorphiDex®, a combination formulation of morphine and dextromethorphan), topiramate, neramexane or perzinfotel including an NR2B antagonist, e.g. ifenprodil, traxoprodil or (−)-(R)-6-{2-[4-(3-fluorophenyl)-4-hydroxy-1-piperidinyl]-1-hydroxyethyl-3,4-dihydro-2(1H)-quinolinone;
an alpha-adrenergic, e.g. doxazosin, tamsulosin, clonidine, guanfacine, dexmetatomidine, modafinil, or 4-amino-6,7-dimethoxy-2-(5-methane-sulfonamido-1,2,3,4-tetrahydroisoquinol-2-yl)-5-(2-pyridyl) quinazoline;
a tricyclic antidepressant, e.g. desipramine, imipramine, amitriptyline or nortriptyline;
an anticonvulsant, e.g. carbamazepine, lamotrigine, topiratmate or valproate;
a tachykinin (NK) antagonist, particularly an NK-3, NK-2 or NK-1 antagonist, e.g. ((αR,9R)-7-[3,5-bis(trifluoromethyl)benzyl]-8,9,10,11-tetrahydro-9-methyl-5-(4-methylphenyl)-7H-[1,4]diazocino[2,1-g][1,7]-naphthyridine-6-13-dione (TAK-637), 5-[[(2R,3S)-2-[(1R)-1-[3,5-bis(trifluoromethyl)phenyl]ethoxy-3-(4-fluorophenyl)-4-morpholinyl]-methyl]-1,2-dihydro-3H-1,2,4-triazol-3-one (MK-869), aprepitant, lanepitant, dapitant or 3-[[2-methoxy-5-(trifluoromethoxy)phenyl]-methylamino]-2-phenylpiperidine (2S,3S);
a muscarinic antagonist, e.g oxybutynin, tolterodine, propiverine, tropsium chloride, darifenacin, solifenacin, temiverine and ipratropium;
a COX-2 selective inhibitor, e.g. celecoxib, rofecoxib, parecoxib, valdecoxib, deracoxib, etoricoxib, or lumiracoxib;
a coal-tar analgesic, in particular paracetamol;
a neuroleptic such as droperidol, chlorpromazine, haloperidol, perphenazine, thioridazine, mesoridazine, trifluoperazine, fluphenazine, clozapine, olanzapine, risperidone, ziprasidone, quetiapine, sertindole, aripiprazole, sonepiprazole, blonanserin, iloperidone, perospirone, raclopride, zotepine, bifeprunox, asenapine, lurasidone, amisulpride, balaperidone, palindore, eplivanserin, osanetant, rimonabant, meclinertant, Miraxion® or sarizotan;
a vanilloid receptor agonist (e.g. resinferatoxin) or antagonist (e.g. capsazepine);
a beta-adrenergic such as propranolol;
a local anaesthetic such as mexiletine;
a corticosteroid such as dexamethasone;
a 5-HT receptor agonist or antagonist, particularly a 5-HT_{1B/1D} agonist such as eletriptan, sumatriptan, naratriptan, zolmitriptan or rizatriptan;
a 5-HT_{2A} receptor antagonist such as R(+)-alpha-(2,3-dimethoxy-phenyl)-1-[2-(4-fluorophenylethyl)]-4-piperidinemethanol (M DL-100907);
a cholinergic (nicotinic) analgesic, such as ispronicline (TC-1734), (E)-N-methyl-4-(3-pyridinyl)-3-buten-1-amine (RJR-2403), (R)-5-(2-azetidinylmethoxy)-2-chloropyridine (ABT-594) or nicotine;
Tramadol®;
a PDEV inhibitor, such as 5-[2-ethoxy-5-(4-methyl-1-piperazinyl-sulphonyl)phenyl]-1-methyl-3-n-propyl-1,6-dihydro-7H-pyrazolo[4,3-d]pyrimidin-7-one (sildenafil), (6R,12aR)-2,3,6,7,12,12a-hexahydro-2-methyl-6-(3,4-methylenedioxyphenyl)-pyrazino[2′,1′:6,1]-pyrido[3,4-b]indole-1,4-dione (IC-351 or tadalafil), 2-[2-ethoxy-5-(4-ethyl-piperazin-1-yl-1-sulphonyl)-phenyl]-5-methyl-7-propyl-3H-imidazo[5,1-f][1,2,4]triazin-4-one (vardenafil), 5-(5-acetyl-2-butoxy-3-pyridinyl)-3-ethyl-2-(1-ethyl-3-azetidinyl)-2,6-dihydro-7H-pyrazolo[4,3-d]pyrimidin-7-one, 5-(5-acetyl-2-propoxy-3-pyridinyl)-3-ethyl-2-(1-isopropyl-3-azetidinyl)-2,6-dihydro-7H-pyrazolo[4,3-d]pyrimidin-7-one, 5-[2-ethoxy-5-(4-ethylpiperazin-1-ylsulphonyl)pyridin-3-yl]-3-ethyl-2-[2-methoxyethyl]-2,6-dihydro-7H-pyrazolo[4,3-d]pyrimidin-7-one, 4-[(3-chloro-4-methoxybenzyl)amino]-2-[(2S)-2-( hydromethyl)pyrrolidin-1-yl]-N-(pyrimidin-2-ylmethyl)pyrimidine-5-carboxamide, 3-(1-methyl-7-oxo-3-propyl-6,7-dihydro-1H-pyrazolo[4,3-d]pyrimidin-5-yl)-N-[2-(1-methylpyrrolidin-2-yl)ethyl]-4-propoxybenzenesulfonamide;
an alpha-2-delta ligand such as gabapentin, pregabalin, 3-methylgabapentin, (1α,3α,5α)(3-amino-methyl-bicyclo[3.2.0]hept-3-yl)-acetic acid, (3S,5R)-3-aminomethyl-5-methyl-heptanoic acid, (3S,5R)-3-amino-5-methyl-heptanoic acid, (3S,5R)-3-amino-5-methyl-octanoic acid, (2S,4S)-4-(3-chlorophenoxy)proline, (2S,4S)-4-(3-fluorobenzyl)-proline, [(1R,5R,6S)-6-(aminomethyl)bicyclo[3.2.0]hept-6-yl]acetic acid, 3-(1-aminomethyl-cyclohexylmethyl)-4H-[1,2,4]oxadiazol-5-one, C-[1-(1H-tetrazol-5-ylmethyl)-cycloheptyl]-methylamine, (3S,4S)-(1-aminomethyl-3,4-dimethyl-cyclopentyl)-acetic acid, (3S,5R)-3-aminomethyl-5-methyl-octanoic acid, (3S,5R)-3-amino-5-methyl-nonanoic acid, (3S,5R)-3-amino-5-methyl-octanoic acid, (3R,4R,5R)-3-amino-4,5-dimethyl-heptanoic acid and (3R,4R,5R)-3-amino-4,5-dimethyl-octanoic acid;
a cannabinoid;
metabotropic glutamate subtype 1 receptor (mGluRl) antagonist;
a serotonin reuptake inhibitor such as sertraline, sertraline metabolite demethylsertraline, fluoxetine, norfluoxetine (fluoxetine desmethyl metabolite), fluvoxamine, paroxetine, citalopram, citalopram metabolite desmethylcitalopram, escitalopram, d,I-fenfluramine, femoxetine, ifoxetine, cyanodothiepin, litoxetine, dapoxetine, nefazodone, cericlamine and trazodone;
a noradrenaline (norepinephrine) reuptake inhibitor, such as maprotiline, lofepramine, mirtazepine, oxaprotiline, fezolamine, tomoxetine, mianserin, buproprion, buproprion metabolite hydroxybuproprion, nomifensine and viloxazine (Vivalan®), especially a selective noradrenaline reuptake inhibitor such as reboxetine, in particular (S,S)-reboxetine;
a dual serotonin-noradrenaline reuptake inhibitor, such as venlafaxine, venlafaxine metabolite O-desmethylvenlafaxine, clomipramine, clomipramine metabolite desmethylclomipramine, duloxetine, milnacipran and imipramine;
an inducible nitric oxide synthase (iNOS) inhibitor such as S-[2-[(1-iminoethyl)amino]ethyl]-L-homocysteine, S-[2-[(1-iminoethyl)-amino]ethyl]-4,4-dioxo-L-cysteine, S-[2-[(1-iminoethyl)amino]ethyl]-2-methyl-L-cysteine, (2S,5Z)-2-amino-2-methyl-7-[(1-iminoethyl)amino]-5-heptenoic acid, 2-[[(1 R,3S)-3-amino-4- hydroxy-1-(5-thiazolyl)-butyl]thio]-5-chloro-3-pyridinecarbonitrile; 2-[[(1 R,3S)-3-amino-4-hydroxy-1-(5-thiazolyl)butyl]thio]-4-chlorobenzonitrile, (2S,4R)-2-amino-4-[[2-chloro-5-(trifluoromethyl)phenyl]thio]-5-thiazolebutanol, 2-[[(1R,3S)-3-amino-4-hydroxy-1-(5-thiazolyl) butyl]thio]-6-(trifluoromethyl)-3 pyridinecarbonitrile, 2-[[(1R,3S)-3- amino-4-hydroxy-1-(5-thiazolyl)butyl]thio]-5-chlorobenzonitrile, N-[4-[2-(3-chlorobenzylamino)ethyl]phenyl]thiophene-2-carboxamidine, or guanidinoethyidisulfide;
an acetylcholinesterase inhibitor such as donepezil;
a prostaglandin E₂ subtype 4 (EP4) antagonist such as N-[({2-[4-(2-ethyl-4,6-dimethyl-1H-imidazo[4,5-c]pyridin-1-yl)phenyl]ethyl}amino)-carbonyl]-4-methylbenzenesulfonamide or 4-[(1S)-1-({[5-chloro-2-(3-fluorophenoxy)pyridin-3-yl]carbonyl}amino)ethyl]benzoic acid;
a leukotriene B4 antagonist; such as 1-(3-biphenyl-4-ylmethyl-4-hydroxy-chroman-7-yl)-cyclopentanecarboxylic acid (CP-105696), 5-[2-(2-Carboxyethyl)-3-[6-(4-methoxyphenyl)-5E-hexenyl]oxyphenoxy]-valeric acid (ONO-4057) or DPC-11870,
a 5-lipoxygenase inhibitor, such as zileuton, 6-[(3-fluoro-5-[4-methoxy-3,4,5,6-tetrahydro-2H-pyran-4-yl])phenoxy-methyl]-1-methyl-2-quinolone (ZD-2138), or 2,3,5-trimethyl-6-(3-pyridylmethyl),1,4-benzoquinone (CV-6504);
a sodium channel blocker, such as lidocaine;
a 5-HT3 antagonist, such as ondansetron;
   and the pharmaceutically acceptable salts and solvates thereof.

Pharmaceutical compositions are suitable for the delivery of compounds of the present invention and methods for their preparation will be readily apparent to those skilled in the art. Such compositions and methods for their preparation may be found, for example, in ‘Remington's Pharmaceutical Sciences’, 19th Edition (Mack Publishing Company, 1995).

### Oral Administration

The compounds of the invention may be administered orally. Oral administration may involve swallowing, so that the compound enters the gastrointestinal tract, or buccal or sublingual administration may be employed by which the compound enters the blood stream directly from the mouth.

Formulations suitable for oral administration include solid formulations such as tablets, capsules containing particulates, liquids, or powders, lozenges (including liquid-filled), chews, multi- and nano-particulates, gels, solid solution, liposome, films (including muco-adhesive), ovules, sprays and and liquid formulations.

Liquid formulations include suspensions, solutions, syrups and elixirs. Such formulations may be employed as fillers in soft or hard capsules and typically comprise a carrier, for example, water, ethanol, polyethylene glycol, propylene glycol, methylcellulose, or a suitable oil, and one or more emulsifying agents and/or suspending agents. Liquid formulations may also be prepared by the reconstitution of a solid, for example, from a sachet.

The compounds of the invention may also be used in fast-dissolving, fast-disintegrating dosage forms such as those described in Expert Opinion in Therapeutic Patents, 11 (6), 981-986 by Liang and Chen (2001).

For tablet dosage forms, depending on dose, the drug may make up from 1 wt % to 80 wt% of the dosage form, more typically from 5 wt % to 60 wt % of the dosage form. In addition to the drug, tablets generally contain a disintegrant. Examples of disintegrants include sodium starch glycolate, sodium carboxymethyl cellulose, calcium carboxymethyl cellulose, croscarmellose sodium, crospovidone, polyvinylpyrrolidone, methyl cellulose, microcrystalline cellulose, lower alkyl-substituted hydroxypropyl cellulose, starch, pregelatinised starch and sodium alginate. Generally, the disintegrant will comprise from 1 wt % to 25 wt %, preferably from 5 wt % to 20 wt % of the dosage form.

Binders are generally used to impart cohesive qualities to a tablet formulation. Suitable binders include microcrystalline cellulose, gelatin, sugars, polyethylene glycol, natural and synthetic gums, polyvinylpyrrolidone, pregelatinised starch, hydroxypropyl cellulose and hydroxypropyl methylcellulose. Tablets may also contain diluents, such as lactose (monohydrate, spray-dried monohydrate, anhydrous and the like), mannitol, xylitol, dextrose, sucrose, sorbitol, microcrystalline cellulose, starch and dibasic calcium phosphate dihydrate.

Tablets may also optionally comprise surface active agents, such as sodium lauryl sulfate and polysorbate 80, and glidants such as silicon dioxide and talc. When present, surface active agents may comprise from 0.2 wt % to 5 wt % of the tablet, and glidants may comprise from 0.2 wt % to 1 wt % of the tablet.

Tablets also generally contain lubricants such as magnesium stearate, calcium stearate, zinc stearate, sodium stearyl fumarate, and mixtures of magnesium stearate with sodium lauryl sulphate. Lubricants generally comprise from 0.25 wt % to 10 wt %, preferably from 0.5 wt % to 3 wt % of the tablet.

Other possible ingredients include anti-oxidants, colourants, flavouring agents, preservatives and taste-masking agents.

Exemplary tablets contain up to about 80% drug, from about 10 wt % to about 90 wt % binder, from about 0 wt % to about 85 wt % diluent, from about 2 wt % to about 10 wt % disintegrant, and from about 0.25 wt % to about 10 wt % lubricant.

Tablet blends may be compressed directly or by roller to form tablets. Tablet blends or portions of blends may alternatively be wet-, dry-, or melt-granulated, melt congealed, or extruded before tabletting. The final formulation may comprise one or more layers and may be coated or uncoated; it may even be encapsulated.

The formulation of tablets is discussed in “Pharmaceutical Dosage Forms: Tablets, Vol. 1”, by H. Lieberman and L. Lachman, Marcel Dekker, N.Y., N.Y., 1980 (ISBN 0-8247-6918-X).

Solid formulations for oral administration may be formulated to be immediate and/or modified controlled release. Modified release formulations include delayed-, sustained-, pulsed-, controlled-, targeted and programmed release.

Suitable modified release formulations for the purposes of the invention are described in U.S. Pat. No. 6,106,864. Details of other suitable release technologies such as high energy dispersions and osmotic and coated particles are to be found in Verma et a/, Pharmaceutical Technology On-line, 25(2), 1-14 (2001). The use of chewing gum to achieve controlled release is described in WO 00/35298.

### Parenteral Administration

The compounds of the invention may also be administered directly into the blood stream, into muscle, or into an internal organ. Suitable means for parenteral administration include intravenous, intraarterial, intraperitoneal, intrathecal, intraventricular, intraurethral, intrasternal, intracranial, intramuscular and subcutaneous. Suitable devices for parenteral administration include needle (including microneedle) injectors, needle-free injectors and infusion techniques.

Parenteral formulations are typically aqueous solutions which may contain excipients such as salts, carbohydrates and buffering agents (preferably, to a pH of from 3 to 9), but, for some applications, they may be more suitably formulated as a sterile non-aqueous solution or as powdered a dried form to be used in conjunction with a suitable vehicle such as sterile, pyrogen-free water.

The preparation of parenteral formulations under sterile conditions, for example, by lyophilisation, may readily be accomplished using standard pharmaceutical techniques well known to those skilled in the art.

The solubility of compounds of formula (I) used in the preparation of parenteral solutions may be increased by the use of appropriate formulation techniques, such as_the incorporation of solubility-enhancing agents. Formulations for use with needle-free injection administration comprise a compound of the invention in powdered form in conjunction with a suitable vehicle such as sterile, pyrogen-free water.

Formulations for parenteral administration may be formulated to be immediate and/or modified controlled release. Modified release formulations include delayed-, sustained-, pulsed-, controlled-, tragetted and programmed release. Thus compounds of the invention may be formulated as a solid, semi-solid, or thixotropic liquid for administration as an implanted depot providing modified release of the active compound. Examples of such formulations include drug-coated stents and PGLA microspheres.

### Topical Administration

The compounds of the invention may also be administered topically to the skin or mucosa, that is, dermally or transdermally. Typical formulations for this purpose to include gels, hydrogels, lotions, solutions, creams, ointments, dusting powders, dressings, foams, films, skin patches, wafers, implants, sponges, fibres, bandages and microemulsions. Liposomes may also be used. Typical carriers include alcohol, water, mineral oil, liquid petrolatum, white petrolatum, glycerin, polyethylene glycol and propylenei glycol. Penetration enhancers may be incorporated - see, for example, J Pharm Sci, 88 (10), 955-958 by Finnin and Morgan (October 1999).

Other means of topical administration include delivery by electroporation, iontophoresis, phonophoresis, sonophoresis and microneedle or needle-free (e.g. Powderject™, Bioject™, etc.) injection.

Formulations for topical administration may be formulated to be immediate and/or modified controlled release. Modified release formulations include delayed-, sustained-, pulsed-, controlled-, tragettedtargeted and programmed release.

### Inhaled/Intranasal Administration

The compounds of the invention can also be administered intranasally or by inhalation, typically in the form of a dry powder (either alone, as a mixture, for example, in a dry blend with lactose, or as a mixed component particle, for example, mixed with phospholipids, such as phosphatidylcholine) from a dry powder inhaler or as an aerosol spray from a pressurised container, pump, spray, atomiser (preferably an atomiser using electrohydrodynamics to produce a fine mist), or nebuliser, with or without the use of a suitable propellant, such as 1,1,1,2-tetrafluoroethane or 1,1,1,2,3,3,3-heptafluoropropane. For intranasal use, the powder may comprise a bioadhesive agent, for example, chitosan or cyclodextrin.

The pressurised container, pump, spray, atomizer, or nebuliser contains a solution or suspension of the compound(s) of the invention comprising, for example, ethanol, aqueous ethanol, or a suitable alternative agent for dispersing, solubilising, or extending release of the active, a propellant(s) as solvent and an optional surfactant, such as sorbitan trioleate, oleic acid, or an oligolactic acid.

Prior to use in a dry powder or suspension formulation, the drug product is micronised to a size suitable for delivery by inhalation (typically less than 5 microns). This may be achieved by any appropriate comminuting method, such as spiral jet milling, fluid bed jet milling, supercritical fluid processing to form nanoparticies, high pressure homogenisation, or spray drying.

Capsules (made, for example, from gelatin or HPMC), blisters and cartridges for use in an inhaler or insufflator may be formulated to contain a powder mix of the compound of the invention, a suitable powder base such as lactose or starch and a performance modifier such as I-leucine, mannitol, or magnesium stearate. The lactose may be anhydrous or in the form of the monohydrate, preferably the latter. Other suitable excipients include dextran, glucose, maltose, sorbitol, xylitol, fructose, sucrose and trehalose.

A suitable solution formulation for use in an atomiser using electrohydrodynamics to produce a fine mist may contain from 1μg to 20 mg of the compound of the invention per actuation and the actuation volume may vary from 1 μl to 100 μl. A typical formulation may comprise a compound of formula (I), propylene glycol, sterile water, ethanol and sodium chloride. Alternative solvents which may be used instead of propylene glycol include glycerol and polyethylene glycol.

Suitable flavours, such as menthol and levomenthol, or sweeteners, such as saccharin or saccharin sodium, may be added to those formulations of the invention intended for inhaled/intranasal administration.

Formulations for inhaled/intranasal administration may be formulated to be immediate and/or modified controlled release using, for example, poly(DL-lactic-coglycolic acid (PGLA). Modified release formulations include delayed-, sustained-, pulsed-, controlled-, targeted and programmed release.

In the case of dry powder inhalers and aerosols, the dosage unit is determined by means of a valve which delivers a metered amount. Units in accordance with the invention are typically arranged to administer a metered dose or “puff” containing from 1 μg to 10 mg of the compound of formula (I). The overall daily dose will typically be in the range 1 μg to 10 mg which may be administered in a single dose or, more usually, as divided doses throughout the day.

### Rectal/Intravaginal Administration

The compounds of the invention may be administered rectally or vaginally, for example, in the form of a suppository, pessary, or enema. Cocoa bufter is a traditional suppository base, but various alternatives may be used as appropriate.

### Ocular/Aural Administration

The compounds of the invention may also be administered directly to the eye or ear, typically in the form of drops of a micronised suspension or solution in isotonic, pH-adjusted, sterile saline. Other formulations suitable for ocular and aural administration include ointments, biodegradable (e.g. absorbable gel sponges, collagen) and non-biodegradable (e.g. silicone) implants, wafers, lenses and particulate or vesicular systems, such as niosomes or liposomes.

### Other Technologies

The compounds of the invention may be combined with soluble macromolecular entities, such as cyclodextrin and suitable derivatives thereof or polyethylene glycol-containing polymers, in order to improve their solubility, dissolution rate, taste-masking, bioavailability and/or stability for use in any of the aforementioned modes of administration.

Drug-cyclodextrin complexes, for example, are found to be generally useful for most dosage forms and administration routes. Both inclusion and non-inclusion complexes may be used. As an alternative to direct complexation with the drug, the cyclodextrin may be used as an auxiliary additive, i.e. as a carrier, diluent, or solubiliser. Most commonly used for these purposes are alpha-, beta- and gamma-cyclodextrins, examples of which may be found in International Patent Applications Nos. WO 91/11172, WO 94/02518 and WO 98/55148.

### Kit-of-Parts

Inasmuch as it may desirable to administer a combination of active compounds, for example, for the purpose of treating a particular disease or condition, it is within the scope of the present invention that two or more pharmaceutical compositions, at least one of which contains a compound in accordance with the invention, may conveniently be combined in the form of a kit suitable for coadministration of the compositions.

Thus the kit of the invention comprises two or more separate pharmaceutical compositions, at least one of which contains a compound of formula (I) in accordance with the invention, and means for separately retaining said compositions, such as a container, divided bottle, or divided foil packet. An example of such a kit is the familiar blister pack used for the packaging of tablets, capsules and the like.

### Dosage

For administration to human patients, the total daily dose of the compounds of the invention is typically in the range 0.1 mg to 3000 mg, preferably from 1 mg to 500 mg, depending, of course, on the mode of administration. For example, oral administration may require a total daily dose of from 0.1 mg to 3000 mg, preferably from l mg to 500 mg, while an intravenous dose may only require from 0.1 mg to 1000 mg, preferably from 0.1 mg to 300 mg. The total daily dose may be administered in single or divided doses.

These dosages are based on an average human subject having a weight of about 65 kg to 70 kg. The physician will readily be able to determine doses for subjects whose weight falls outside this range, such as infants and the elderly.

For the avoidance of doubt, references herein to “treatment” include references to curative, palliative and prophylactic treatment.

### EXAMPLES

The invention is illustrated in the following non-limiting examples in which, unless stated otherwise: all operations were carried out at room or ambient temperature, that is, in the range of 18-25° C.; evaporation of solvent was carried out using a rotary evaporator under reduced pressure with a bath temperature of up to 60° C.; reactions were monitored by thin layer chromatography (TLC); the structure and purity of all isolated compounds were assured by at least one of the following techniques: TLC (Merck silica gel 60 F₂₅₄ precoated TLC plates or Merck NH₂ gel (an amine coated silica gel) F₂₅4s precoated TLC plates), mass spectrometry, nuclear magnetic resonance spectra (NMR) or infrared red absorption spectra (IR). Yields are given for illustrative purposes only. Workup with a cation-exchange column was carried out using SCX cartridge (Varian BondElute), which was preconditioned with methanol. Flash column chromatography was carried out using Merck silica gel 60 (63-200 μm), Wako silica gel 300 HG (40-60 μm), Fuji Silysia NH gel (an amine coated silica gel) (30-50 μm), Biotage KP-SIL (32-63 μm) or Biotage AMINOSILICA (an amine coated silica gel) (40-75 μm). Preparative TLC was carried out using Merck silica gel 60 F₂₅₄ precoated TLC plates (0.5 or 1.0 mm thickness). Low-resolution mass spectral data (EI) were obtained on an Integrity (Waters) mass spectrometer. Low-resolution mass spectral data (ESI) were obtained on a ZMD (Micromass) mass spectrometer. NMR data was determined at 270 MHz (JEOL JNM-LA 270 spectrometer), 300 MHz (JEOL JNM-LA300 spectrometer) or 600 MHz (Bruker AVANCE 600 spectrometer) using deuterated chloroform (99.8% D) or dimethylsulfoxide (99.9% D) as solvent unless indicated otherwise, relative to tetramethylsilane (TMS) as internal standard in parts per million (ppm); conventional abbreviations used are: s=singlet, d=doublet, t=triplet, q=quartet, quint=quintet, m=multiplet, br.=broad, etc. IR spectra were measured by a Shimazu infrared spectrometer (IR-470). Chemical symbols have their usual meanings; L (liter(s)), mL (milliliter(s)), g (gram(s)), mg (milligram(s)), mol (moles), mmol (millimoles), eq. (equivalent(s)), quant. (quantitative yield), min (minute(s)), h (hour(s)).

### Example 1

### 3-(3′H,8H-SPIRO[8-AZABICYCLO[3.2.1]OCTANE-3,1′-[2]BENZOFURAN]-8-YL)-2-(1,3-THIAZOL-4-YLMETHYL)PROPANOIC ACID TRIFLUOROACETATE

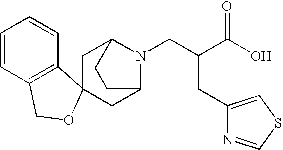

### STEP 1. tert-Butyl 2-(diethoxyphosphoryl)-3-(1,3-thiazol-4-yl)propanoate

A mixture of 4-methylthiazole (5.85 g, 59 mmol), N-bromosuccinimide (11 g, 62 mmol) and 2,2′-azobisisobutyronitrile (968 mg, 5.9 mmol) in carbontetrachloride (200 mL) was refluxed for 5 hours. After cooling, the mixture was filtered. To the filtrate was added toluene (100 mL) and the mixture was concentrated to afford a toluene solution of 4-(bromomethyl)-1,3-thiazole (27 g).

To a solution of tert-butyl diethylphosphonoacetate (15.6 g, 62 mmol) in dimethylformamide (50 mL) was added sodium hydride (60% dispersion in mineral oil, 2.48 g, 62 mmol) at 0° C. under a nitrogen atmosphere. After 45 minutes, a solution of 4-(bromomethyl)-1,3-thiazole in toluene (27 g) was added to the mixture and the mixture was stirred at room temperature overnight. The mixture was quenched with water and extracted with toluene/ethyl acetate (1/3). The combined organic layer was washed with brine, dried over sodium sulfate, and evaporated. The residue was purified by column chromatography on silica gel, eluting with hexane/ethyl acetate (1/2 to 100% ethyl acetate), to afford 7.17 g (35%) of the title compound as a colorless oil:

¹H-NMR (CDCl₃) δ 8.74 (1H, d, J=2.0 Hz), 7.06 (1H, d, J=1.8 Hz), 4.24-4.08 (4H, m), 3.55-3.24 (3H, m), 1.45-1.30 (15H, m).

### STEP 2. tert-Butyl 2-(1,3-thiazol-4-ylmethyl)acrylate

To a stirred solution of tert-butyl 2-(diethoxyphosphoryl)-3-(1,3-thiazol-4-yl)propanoate (step 1, 7.17 g, 20.5 mmol) in tetrahydrofuran (100 mL) was added sodium hydride (60% dispersion in mineral oil, 820 mg, 20.5 mmol) at 0° C. under nitrogen. After 10 minutes, to the mixture was added paraformaldehyde (1.85 g, 61.5 mmol) and the mixture was stirred at room temperature for 45 minutes. The mixture was quenched with aqueous sodium hydrogen carbonate and extracted with ethyl acetate. The combined organic layer was washed with brine, dried over sodium sulfate, and evaporated. The residue was purified by column chromatography on silica gel, eluting with hexane/ethyl acetate (3/1), to afford 4.25 g (92%) of the title compound as a colorless oil:

¹H-NMR (CDCl₃) δ 8.77 (1H, d, J=2.0 Hz), 7.04 (1H, d, J=2.0 Hz), 6.23-6.20 (1H, m), 5.52 (1H, q, J=1.3 Hz), 3.83 (2H, s), 1.44 (9H, s); MS (ESI) 226 (M+H)⁺.

### STEP 3. tert-Butyl 3-(3′H,8H-spiro[8-azabicyclo[3.2.1]octane-3,1′-[2]benzofuran]-8-yl)-2-(1,3-thiazol-4-ylmethyl)propanoate

A solution of 3′H-spiro[8-azabicyclo[3.2.1]octane-3,1′-[2]benzofuran] (Bioorg. Med. Chem. Lett. 1998, 8, 1541. 150 mg, 0.7 mmol) and tert-butyl 2-(1,3-thiazol-4-ylmethyl)acrylate (step 2, 157 mg, 0.7 mmol) in methanol (1 mL) was stirred at room temperature for 3 days. The reaction mixture was evaporated to give a slight yellow syrup. The residue was purified by column chromatography on silica gel (40 g), eluting with hexane/ethyl acetate (3/1), to afford 69.1 mg (22%) of the title compound as a colorless syrup:

¹H-NMR (CDCl₃) δ 8.75 (1H, d, J=1.8 Hz), 7.23-7.15 (3H, m), 7.05-7.02 (2H, m), 4.99 (2H, s), 3.33-3.21 (2H, m), 3.10-2.94 (3H, m), 2.72-2.56 (2H, m), 2.21-2.15 (2H, m), 2.09-2.03 (2H, m), 1.88-1.76 (4H, m), 1.40 (9H, s); MS (ESI) 441 (M+H)⁺.

### STEP 4. 3-(3′H,8H-Spiro[8-azabicyclo[3.2.1]octane-3,1′-[2]benzofuran]-8-yl)-2-(1,3-thiazol-4-ylmethyl)propanoic acid trifluoroacetate

To a stirred solution of tert-butyl 3-(3′H,8H-spiro[8-azabicyclo[3.2.1]octane-3,1′-[2]benzofuran]-8-yl)-2-(1,3-thiazol-4-ylmethyl)propanoate (step 3) in dichloromethane (2 mL) was added trifluoroacetic acid (2 mL) and the mixture was stirred at room temperature for 2 hours. The reaction mixture was evaporated to dryness to afford the title compound as a yellow oil (85.3 mg, 100%): MS (ESI) 385 (M+H)⁺.

### Example 2

### 3-(1H-PYRAZOL-1-YL)-2-(3′H,8H-SPIRO[8-AZABICYCLO[3.2.1]OCTANE-3,1′-[2]BENZOFURAN]-8-YLMETHYL)PROPANOIC ACID

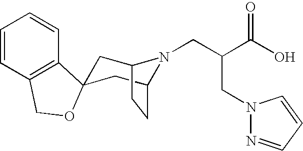

### STEP 1. Ethyl 2-(1H-pyrazol-1-ylmethyl)acrylate

A mixture of ethyl 2-(hydroxymethyl)acrylate (4.1 g, 32 mmol), pyrazole (2.6 g, 38 mmol) and potassium carbonate (11 g, 79 mmol) in acetonitrile (30 mL) was refluxed for 20 hours, quenched by the addition of water (100 mL), and extracted with ethyl acetate (40 mL×2). The combined organic layers were washed with brine, dried over magnesium sulfate, and evaporated. The residue was purified by column chromatography on silica gel, eluting with hexane/ethyl acetate (7/1), to afford 1.0 g (18%) of the title compound as a colorless oil:

¹H-NMR (CDCl₃) δ 7.57-7.53 (1H, m), 7.48-7.45 (1H, m), 6.36-6.32 (1H, m), 6.28 (1H, t, J=2.0 Hz), 5.48-5.44 (1H, m), 5.01 (2H, s), 4.24 (2H, q, J=7.1 Hz), 1.30 (3H, t, J=7.1 Hz).

### STEP 2. Ethyl

### 3-(1H-Pyrazol-1-yl)-2-(3′H,8H-spiro[8-azabicyclo[3.2.1]octane-3,1′-[2]benzofuran]-8-ylmethyl)propanoate

The title compound was prepared from 3′H-spiro[8-azabicyclo[3.2.1]octane-3,1′-[2]benzofuran] (Bioorg. Med. Chem. Lett. 1998, 8, 1541.) and ethyl 2-(1H-pyrazol-1-ylmethyl)acrylate (step 1) according to the procedure described in step 3 of example 1:

¹H-NMR (CDCl₃) δ 7.52 (1H, d, J=1.7 Hz), 7.42 (1H, d, J=2.2 Hz), 7.26-7.16 (3H, m), 7.08-7.04 (1H, m), 6.22 (1H, t, J=1.7 Hz), 5.00 (2H, s), 4.55-4.42 (2H, m), 4.15 (2H, q, J=7.2 Hz), 3.24-3.15 (3H, m), 2.70-2.57 (2H, m), 2.24-2.17 (2H, m), 2.09-2.00 (2H, m), 1.91-1.78 (4H, m), 1.23 (3H, t, J=7.1 Hz);

MS (ESI) 396 (M+H)⁺.

### STEP 3. 3-(1H-Pyrazol-1-yl)-2-(3′H,8H-spiro[8-azabicyclo[3.2.1]octane-3,1′-[2]benzofuran]-8-ylmethyl)propanoic acid

To a stirred solution of ethyl 3-(1H-pyrazol-1-yl)-2-(3′H,8H-spiro[8-azabicyclo[3.2.1]octane-3,1′-[2]benzofuran]-8-ylmethyl)propanoate (step 2, 45.0 mg, 0.114 mmol) in tetrahydrofuran (1 mL) and methanol (1 mL) was added 2 N sodium hydroxide aqueous solution (1 mL) at room temperature. The reaction mixture was stirred at room temperature for 14 hours, evaporated to remove methanol, and acidified with sodium hydrogenphosphate aqueous solution to pH 4-5. The aqueous layer was extracted with ethyl acetate. The organic layer was washed with brine, dried over magnesium sulfate, and evaporated to afford the title compound as a white solid: MS (ESI) 368 (M+H)⁺, 366 (M−H)⁻.

### Example 3

### 6′-FLUORO-3′H,8H-SPIRO[8-AZABICYCLO[3.2.1]OCTANE-3,1′-[2]BENZOFURAN[-8-CARBOXYLATE

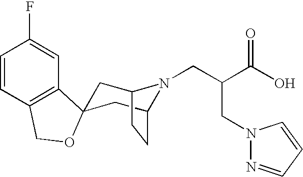

### STEP 1. (2-Bromo-4-fluorophenyl)methanol

To a stirred solution of 2-bromo-4-fluorobenzoic acid (8.0 g, 37 mmol) in tetrahydrofuran (150 mL) was added dropwise borane-methyl sulfide complex (8.7 mL, 91 mmol) at 0° C., and the mixture was stirred for 2 hours at room temperature. Another 3.0 mL (32 mmol) borane-methyl sulfide complex was added to the reaction mixture at room temperature. The mixture was warmed to 60 ° C. for 3 hours with stirring then cooled to 0° C., quenched by the addition of 2N hydrogen chloride aqueous solution (100 mL), stirred for 30 minutes, and extracted with ethyl acetate. The extracts were combined, washed with brine, dried over magnesium sulfate, and evaporated. The residue was purified by column chromatography on silica gel, eluting with hexane/ethyl acetate (4/1), to afford 6.8 g (90%) of the title compound as a white solid:

¹H-NMR (CDCl₃) δ8.47 (1H, dd, J=8.6, 6.1 Hz), 7.31 (1H, dd, J=8.3, 2.6 Hz), 7.10-7.02 (1H, m), 4.72 (2H, d, J=6.2 Hz), 1.99 (1H, t, J=6.2 Hz).

### STEP 2. Ethyl 3-[5-fluoro-2-(hydroxymethyl)phenyl]-3-hydroxy-8-azabicyclo[3.2.1]octane-8-carboxylate

To a stirred solution of (2-bromo-4-fluorophenyl)methanol (10 g, 49 mmol, step1) in tetrahydrofuran (50 mL) and toluene (50 mL) was added dropwise a 1.58 M solution of butyllithium in hexane (65 mL, 100 mmol) at −78° C. for 1 hour and the mixture was stirred for 2 hours at the same temperature. To the mixture was added dropwise a solution of ethyl 3-oxo-8-azabicyclo[3.2.1]octane-8-carboxylate in tetrahydrofuran (10 mL) at −78° C. for 10 minutes. This resulting mixture was slowly warmed up to room temperature and stirred for 19 hours at the same temperature. The reaction mixture was quenched by the addition of saturated ammonium chloride aqueous solution, and extracted with ethyl acetate. The organic layer was separated, washed with brine, dried over magnesium sulfate, and evaporated. The residue was purified by column chromatography on silica gel, eluting with hexane/ethyl acetate (2/1), to afford 7.1 g (45%) of the title compound as a white solid:

¹H-NMR (CDCl₃) δ 7.19 (1H, dd, J=8.4, 6.1 Hz), 6.98 (1H, dd, J=11.2, 2.6 Hz), 6.90-6.80 (1H, m), 4.79 (2H, s), 4.43-4.30 (2H, m), 4.25-4.06 (3H, m), 3.31 (1H, s), 2.50-2.22 (4H, m), 2.05-1.85 (4H, m), 1.28 (3H, t, J=7.3 Hz); MS (ESI) 322 (M−H)⁻.

### STEP 3. Ethyl 6′-fluoro-3′H,8H-spiro[8-azabicyclo[3.2.1]octane-3,1′-[2]benzofuran]-8-carboxylate

To a stirred solution of ethyl 3-[5-fluoro-2-(hydroxymethyl)phenyl]-3-hydroxy-8-azabicyclo[3.2.1]octane-8-carboxylate (7.1 g, 22 mmol, step 2) and triethylamine (9.2 mL, 66 mmol dichloromethane (70 mL) was added dropwise methanesulfonyl chloride (2.1 mL, 27 mmol) at 0° C. This resulting mixture was slowly warmed up to room temperature and stirred for 1 hour at the same temperature. The reaction mixture was washed with sodium hydrogen carbonate aqueous solution, dried over magnesium sulfate, and evaporated. The residue was purified by column chromatography on silica gel, eluting with hexane/ethyl acetate (10/1), to afford 5.8 g (85%) of the title compound as a white solid:

¹H-NMR (CDCl₃) δ 7.12 (1H, dd, J=8.3, 5.0 Hz), 6.98-6.88 (1H, m), 6.70 (1H, dd, J=8.6, 2.2 Hz), 5.00 (2H, s), 4.47-4.14 (4H, m), 2.37-2.24 (2H, m), 2.20-1.85 (6H, m), 1.31 (3H, t, J=7.3 Hz);

MS (ESI) 306 (M+H)⁺.

### STEP 4. 6′-Fluoro-3′H-spiro[8-azabicyclo[3.2.1]octane-3,1′-[2]benzofuran]

A solution of ethyl 6′-fluoro-3′H,8H-spiro[8-azabicyclo[3.2.1]octane-3,1′-[2]benzofuran]-8-carboxylate (3.2 g, 11 mmol, step 3) in 40% sodium hydroxide aqueous solution (20 mL) and ethanol (30 mL) was refluxed for 3 days. The reaction mixture was concentrated to remove ethanol. The crude material was partitioned between diethyl ether and water, and the organic layer was washed with brine, dried over magnesium sulfate, and evaporated to afford 2.2 g (91%) of the title compound as a pale brown solid: MS (ESI) 234 (M+H)⁺.

### STEP 5. Ethyl 3-(6′-fluoro-3′H,8H-spiro[8-azabicyclo[3.2.1]octane-3,1′-[2]benzofuran]-8-yl)-2-(1H-pyrazol-1-ylmethyl)propanoate

The title compound was prepared from 6′-fluoro-3′H-spiro[8-azabicyclo[3.2.1]octane-3,1′-[2]benzofuran] (step 4) and ethyl 2-(1H-pyrazol-1-ylmethyl)acrylate (step 1 of example 2) according to the procedure described in step 3 of example 1:

¹H-NMR (CDCl₃) δ 7.53 (1H, d, J=1.8 Hz), 7.42 (1H, d, J=2.2 Hz), 7.14-7.06 (1H, m), 6.96-6.86 (1H, m), 6.77-6.69 (1H, m), 6.25-6.18 (1H, m), 4.95 (2H, s), 4.56-4.40 (2H, m), 4.15 (2H, q, J=7.2 Hz), 3.28-3.13 (3H, m), 2.70-2.54 (2H, m), 2.25-2.13 (2H, m), 2.07-1.94 (2H, m), 1.92-1.77 (4H, m), 1.24 (3H, t, J=7.2 Hz); MS (ESI) 414 (M+H)⁺.

### STEP 6. 3-(6′-Fluoro-3′H,8H-spiro[8-azabicyclo[3.2.1]octane-3,1′-[2]benzofuran]-8-yl)-2-(1H-pyrazol-1-ylmethyl)propanoic acid

The title compound was prepared from ethyl 3-(6′-fluoro-3′H,8H-spiro[8-azabicyclo[3.2.1]octane-3,1′-[2]benzofuran]-8-yl)-2-(1H-pyrazol-1-ylmethyl)propanoate (step 5) according to the procedure described in step 3 of example 2: MS (ESI) 386 (M+H)⁺, 384 (M−H)⁻.

### Example 4

### 3-(6′-FLUORO-3′H,8H-SPIRO[8-AZABICYCLO[3.2.1]OCTANE-3,1′-[2]BENZOFURAN]-8-YL)-2-(1,3-THIAZOL-4-YLMETHYL)PROPANOIC ACID TRIFLUOROACETATE

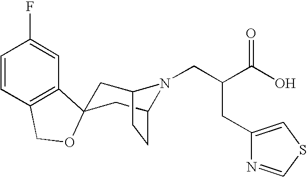

### STEP 1. tert-Butyl 3-(6′-fluoro-3′H,8H-spiro[8-azabicyclo[3.2.1]octane-3,1′-[2]benzofuran]-8-yl)-2-(1,3-thiazol-4-ylmethyl)propanoate

The title compound was prepared from 6′-fluoro-3′H-spiro[8-azabicyclo[3.2.1]octane-3,1′-[2]benzofuran] (step 4 of example 3) and tert-butyl 2-(1,3-thiazol-4-ylmethyl)acrylate (step 2 of example 1) according to the procedure described in step 3 of example 1:

¹H-NMR (CDCl₃) δ 8.76 (1H, d, J=2.0 Hz), 7.14-7.05 (1H, m), 7.03 (1H, d, J=2.0 Hz), 6.95-6.85 (1H, m), 6.74-6.66 (1H, m), 4.94 (2H, s), 3.34-3.20 (2H, m), 3.12-2.90 (3H, m), 2.74-2.53 (2H, m), 2.22-2.10 (2H, m), 2.07-1.95 (2H, m), 1.92-1.74 (4H, m), 1.41 (9H, s); MS (ESI) 459 (M+H)⁺.

### STEP 2. 3-(6′-Fluoro-3′H,8H-spiro[8-azabicyclo[3.2.1]octane-3,1′-[2]benzofuran]-8-yl)-2-(1,3-thiazol-4-ylmethyl)propanoic acid trifluoroacetate

The title compound was prepared from tert-butyl 3-(6′-fluoro-3′H,8H-spiro[8-azabicyclo[3.2.1]octane-3,1′-[2]benzofuran]-8-yl)-2-(1,3-thiazol-4-ylmethyl)propanoate (step 1) according to the procedure described in step 4 of example 1: MS (ESI) 403 (M+H)⁺, 401 (M−H)⁻.

### Example 5

### 3-(3′,4′-DIHYDRO-8H-SPIRO[8-AZABICYCLO[3.2.1]OCTANE-3,1′-lSOCHROMEN]-8-YL)-2-(1H-PYRAZOL-1-YLMETHYL)PROPANOIC ACID

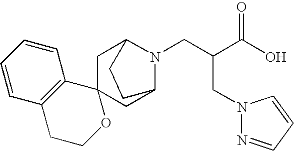

### STEP 1. Ethyl 3-hydroxy-3-[2-(2-hydroxyethyl)phenyl]-8-azabicyclo[3.2.1]octane-8-carboxylate

The title compound was prepared from 2-(2-bromophenyl)ethanol and ethyl 3-oxo-8-azabicyclo[3.2.1]octane-8-carboxylate according to the procedure described in step 2 of example 3:

¹H-NMR (CDCl₃) δ 7.55-7.46 (1H, m), 7.30-7.10 (3H, m), 4.47-4.34 (2H, m), 4.22 (2H, q, J=7.2 Hz), 3.88-3.76 (2H, m), 3.18-1.65 (10H, m), 1.30 (3H, t, J=7.2 Hz); MS (ESI) 320 (M+H)⁺.

### STEP 2. Ethyl 3′,4′-dihydro-8H-spiro[8-azabicyclo[3.2.1]octane-3,1′-isochromene]-8-carboxylate

The title compound was prepared from ethyl 3-hydroxy-3-[2-(2-hydroxyethyl)phenyl]-8-azabicyclo[3.2.1]octane-8-carboxylate (step 1) according to the procedure described in step 3 of example 3:

¹H-NMR (CDCl₃) δ 7.19-6.94 (4H, m), 4.42-4.10 (4H, m), 3.87 (2H, q, J=7.2 Hz), 2.79 (2H, t, J=5.5 Hz), 2.31-1.80 (8H, m), 1.32 (3H, t, J=7.2 Hz); MS (ESI) 302 (M+H)⁺.

### STEP 3. 3′,4′-Dihydrospiro[8-azabicyclo[3.2.1]octane-3,1′-isochromene]

The title compound was prepared from ethyl 3′,4′-dihydro-8H-spiro[8-azabicyclo[3.2.1]octane-3,1′-isochromene]-8-carboxylate (step 2) according to the procedure described in step 4 of example 3:

¹H-NMR (CDCl₃) δ 7.23-7.00 (4H, m), 3.85 (2H, t, J=5.7 Hz), 3.64-3.55 (2H, m), 2.78 (2H, t, J=5.7 Hz), 2.27-2.20 (2H, m), 2.10-1.71 (6H, m); MS (ESI) 230 (M+H)⁺.

### STEP 4. Ethyl 3-(3′,4′-dihydro-8H-spiro[8-azabicyclo[3.2.1]octane-3,1′-isochromen]-8-yl)-2-(1H-pyrazol-1-ylmethyl)propanoate

The title compound was prepared from 3′,4′-dihydrospiro[8-azabicyclo[3.2.1]octane-3,1′-isochromene] (step 3) and ethyl 2-(1H-pyrazol-1-yl)acrylate (step 1 of example 2) according to the procedure described in step 3 of example 1:
¹H-NMR (CDCl₃) δ 7.54-7.50 (1H, m), 7.45-7.42 (1H, m), 7.22-7.05 (3H, m), 7.03-6.98 (1H, m), 6.25-6.20 (1H, m), 4.58-4.44 (2H, m), 4.16 (2H, q, J=6.6 Hz), 3.86-3.78 (2H, m), 3.25-3.16 (3H, m), 2.80-2.73 (2H, m), 2.67-2.60 (2H, m), 2.18-1.95 (6H, m), 1.87-1.76 (2H, m), 1.23 (3H, t, J=6.6 Hz);

MS (ESI) 410(M+H)⁺.

### STEP 5. 3-(3′,4′-Dihydro-8H-spiro[8-azabicyclo[3.2.1]octane-3,1′-isochromen]1-8-yl)-2-(1H-pyrazol-1-ylmethyl)propanoic acid

The title compound was prepared from ethyl 3-(3′,4′-dihydro-8H-spiro[8-azabicyclo[3.2.1]octane-3,1′-isochromen]-8-yl)-2-(1H-pyrazol-1-ylmethyl)propanoate (step 4) according to the procedure described in step 3 of example 2: MS (ESI) 382 (M+H)⁺, 380 (M−H)⁻.

### Example 6

### 3-(6′-FLUORO-3′,4′-DIHYDRO-8H-SPIRO[8-AZABICYCLO[3.2.1]OCTANE-3,1′-ISOCHROMEN]8-YL)-2-(1H-PYRAZOL-1-YLMETHYL)PROPANOIC ACID

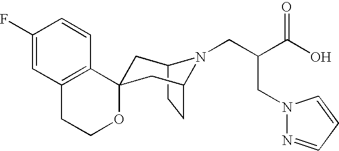

### STEP 1. 2-(2-Bromo-5-fluorophenyl)ethanol

To a solution of (2-bromo-5-fluorophenyl)acetic acid (1.29 g, 5.54 mmol) in tetrahydrofuran (15 mL) was added lithium aluminum hydride (210 mg, 5.54 mmol) at 0 ° C. The mixture was warmed to room temperature and stirred for 3 hours. After cooling to 0 ° C., the reaction mixture was quenched by the addition of 2N hydrochloric acid (30 mL) and extracted with diethyl ether (200 mL). The organic layer was washed with water (50 mL) and brine (50 mL), dried over magnesium sulfate, and evaporated. The residue was purified by column chromatography on silica gel (40 g), eluting with hexane/ethyl acetate (5/1), to afford 247 mg (20%) of the title compound as a colorless oil:

¹H-NMR (CDCl₃) δ7.51 (1H, dd, J=8.8, 5.4 Hz), 7.04 (1H, dd, J=9.2, 3.1 Hz), 6.84 (1H, dt, J=8.4, 3.1 Hz), 3.93-3.87 (2H, m), 3.01 (2H, t, J=6.6 Hz), 1.44 (1H, t, J=5.7 Hz).

### STEP 2. Ethyl 3-[4-fluoro-2-(2-hydroxyethyl)phenyl]-3-hydroxy-8-azabicyclo[3.2.1]octane-8-carboxylate

The title compound was prepared from 2-(2-bromo-5-fluorophenyl)ethanol (step 1) and ethyl 3-oxo-8-azabicyclo[3.2.1]octane-8-carboxylate according to the procedure described in step 2 of example 3:

¹H-NMR (CDCl₃) δ 7.55-7.45 (1H, m), 6.95-6.75 (2H, m), 4.50-4.30 (2H, m), 4.23 (2H, q, J=7.3 Hz), 3.90-3.75 (2H, m), 3.20-2.75 (2H, m), 2.70-2.20 (4H, m), 2.10-1.95 (2H, m), 1.85-1.70 (2H, m), 1.31 (3H, t, J=7.3 Hz).

### STEP 3. Ethyl 6′-fluoro-3′,4′-dihydro-8H-spiro[8-azabicyclo[3.2.1]octane-3,1′-isochromene]-8-carboxylate

The title compound was prepared from ethyl 3-[4-fluoro-2-(2-hydroxyethyl)phenyl]-3-hydroxy-8-azabicyclo[3.2.1]octane-8-carboxylate (step 2) according to the procedure described in step 3 of example 3:

¹H-NMR (CDCl₃) δ 6.98-6.80 (2H, m), 6.78-6.70 (1H, m), 4.45-4.10 (4H, m), 3.87 (2H, t, J=5.5 Hz), 2.78 (2H, t, J=5.5 Hz), 2.30-1.80 (8H, m), 1.32 (3H, t, J=7.2 Hz); MS (ESI) 320 (M+H)⁺.

### STEP 4. 6′-Fluoro-3′,4′-dihydrospiro[8-azabicyclo[3.2.1]octane-3,1′-isochromene]

The title compound was prepared from ethyl 6′-fluoro-3′,4′-dihydro-8H-spiro[8-azabicyclo[3.2.1]octane-3,1′-isochromene]-8-carboxylate (step 3) according to the procedure described in step 4 of example 3:

¹H-NMR (CDCl₃) δ 7.18 (1H, dd, J=8.8, 5.5 Hz), 6.88 (1H, dt, J=8.8, 2.8 Hz), 6.72 (1H, dd, J=9.2, 2.8 Hz), 3.84 (2H, t, J=5.5 Hz), 3.65-3.55 (2H, m), 2.76 (2H, t, J=5.5 Hz), 2.30-1.65 (8H, m);

MS (ESI) 248 (M+H)⁺.

### STEP 5. Ethyl 3-(6′-fluoro-3′,4′-dihydro-8H-spiro[8-azabicyclo[3.2.1]octane-3,1′-isochromen]-8-yl)-2-(1H-pyrazol-1-ylmethyl)propanoate

The title compound was prepared from 6′-fluoro-3′,4′-dihydrospiro[8-azabicyclo[3.2.1]octane-3,1′-isochromene] (step 4) and ethyl 2-(1H-pyrazol-1-ylmethyl)acrylate (step 1 of example 2) according to procedure described in step 3 of example 1:

¹H-NMR (CDCl₃) δ 7.53 (1H, d, J=1.8 Hz), 7.43 (1H, d, J=1.8 Hz), 7.07 (1H, dd, J=8.8, 5.5 Hz), 6.87 (1H, dt, J=8.8, 2.8 Hz), 6.70 (1H, dd, J=9.2, 2.8 Hz), 6.22 (1H, t, J=1.8 Hz), 4.60-4.40 (2H, m), 4.15 (2H, q, J=7.2 Hz), 3.81 (2H, t, J=5.5 Hz), 3.25-3.13 (3H, m), 2.74 (2H, t, J=5.5 Hz), 2.70-2.55 (2H, m), 2.15-1.60 (8H, m), 1.23 (3H, t, J=7.2 Hz); MS (ESI) 428 (M+H)⁺.

### STEP 6. 3-(6′-Fluoro-3′,4′-dihydro-8H-spiro[8-azabicyclo[3.2.1]octane-3,1′-isochromen]-8-yl)-2-(1H-pyrazol-1-ylmethyl)propanoic acid

The title compound was prepared from ethyl 3-(6′-fluoro-3′,4′-dihydro-8H-spiro[8-azabicyclo[3.2.1]octane-3,1′-isochromen]-8-yl)-2-(1H-pyrazol-1-ylmethyl)propanoate (step 5) according to the procedure described in step 3 of example 2: MS (ESI) 400 (M+H)⁺, 398 (M−H)⁻.

### Example 7

### 2-(2-CHLOROBENZYL)-3-(6′-FLUORO-3′,4′-DIHYDRO-8H-SPIRO[8-AZABICYCLO]3.2.1]OCTANE-3,1′-ISOCHROMEN]1-8-YL)PROPANOIC ACID

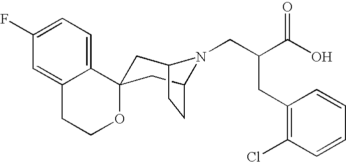

### STEP 1. Ethyl 3-(2-chlorophenyl)-2-(diethoxyphosphoryl)propanoate

To a stirred solution of ethyl (diethoxyphosphoryl)acetate (10.0 g, 44.6 mmol) in N,N-dimethylformamide (100 mL) was added 60% sodium hydride in mineral oil (1.96 g, 49.1 mmol) at 0° C. and the mixture was stirred for 1 hour at the same temperature. To the mixture was added 1-(bromomethyl)-2-chlorobenzene (6.35 mL, 49.1 mmol) at 0° C. and the resulting mixture was stirred for 18 hours at room temperature. The reaction mixture was quenched by the addition of water, then extracted with diethyl ether (200 mL×2), and the combined organic layers were washed with water (100 mL) and brine (100 mL), dried over sodium sulfate, and evaporated. The residue was purified by column chromatography on silica gel (500 g), eluting with hexane/ethyl acetate (1/1), to afford 14.6 g (93%) of the title compound as a colorless oil:

¹H-NMR (CDCl₃) δ 7.36-7.09 (4H, m), 4.26-4.06 (6H, m), 3.52-3.27 (3H, m), 1.39-1.33 (6H, m), 1.15 (3H, t, J=7.0 Hz).

### STEP 2 Ethyl 2-(2-chlorobenzyl)acrylate

To a stirred mixture of ethyl 3-(2-chlorophenyl)-2-(diethoxyphosphoryl)propanoate (step 1, 14.6 g, 41.9 mmol) and 37% formaldehyde in water (20 mL) was added a solution of potassium carbonate (17.4 g) in water (80 mL) at room temperature and the mixture was stirred for 6 hours at 90° C. After cooling to room temperature, the mixture was extracted with diethyl ether (300 mL), and then the organic layer was washed with brine (100 mL), dried over magnesium sulfate, and evaporated. The residue was purified by column chromatography on silica gel (300 g), eluting with hexane/ethyl acetate (30/1), to afford 6.57 g (70%) of the title compound as a colorless oil:

¹H-NMR (CDCl₃) δ 7.39-7.36 (1 H, m), 7.25-7.16 (3H, m), 6.27 (1H, q, J=1.3 Hz), 5.33 (1H, q, J=1.7 Hz), 4.22 (2H, q, J=7.2 Hz), 3.76 (2H, t, J=1.4 Hz), 1.29 (3H, t, J=6.0 Hz).

### STEP 3. Ethyl 2-(2-chlorobenzyl)-3-(6′-fluoro-3′,4′-dihydro-8H-spiro[8-azabicyclo[3.2.1]octane-3,1′-isochromen]-8-yl)propanoate

A solution of 6′-fluoro-3′,4′-dihydrospiro[8-azabicyclo[3.2.1]octane-3,1′-isochromene] (step 4 of example 6, 683.1 mg, 2.76 mmol) and ethyl 2-(2-chlorobenzyl)acrylate (step 2, 564.2 mg, 2.51 mmol) in ethanol (2.0 mL) was stirred at 25° C. for 5 days. The reaction mixture was concentrated in vacuo to give brown syrup. The residue was purified by column chromatography on silica gel, eluting with hexane/ethyl acetate (6/1), to give the title product which contained a small amount of impurity. Then, the product was further purified by preparative TLC on silica gel, developing with CH₂Cl₂/MeOH (60/1), to afford 476.9 mg (40.3%) of the title compound as a colorless oil:

¹H-NMR (300 MHz, CDCl₃, ppm) δ7.38-7.32 (1H, m), 7.27-7.24 (1H, m), 7.20-7.13 (2H, m), 7.04 (1H, dd, J=8.8 Hz, 6.0 Hz), 6.83 (1H, ddd, J=8.8 Hz, 8.8 Hz, 2.9 Hz), 6.75 (1H, dd, J=8.8 Hz, 2.9 Hz), 4.09 (2H, q, J=7.3 Hz), 3.81 (2H, t, J=5.1 Hz), 3.30-3.19 (3H, m), 3.02-2.89 (2H, m), 2H, m), 2.75-2.68 (3H, m), 2.89-2.53 (1H, m), 2.11-1.76 (8H, m), 1.17 (3H, t, J=7.3 Hz); MS (ESI positive) m/z: 472 (M+H)⁺.

### STEP 4. 2-(2-Chlorobenzyl)-3-(6′-fluoro-3′,4′-dihydro-8H-spiro[8-azabicyclo[3.2.1]octane-3,1′-isochromen]-8-yl)propanoic acid

To a stirred solution of ethyl 2-(2-chlorobenzyl)-3-(6′-fluoro-3′,4′-dihydro-8H-spiro[8-azabicyclo[3.2.1]octane-3,1′-isochromen]-8-yl)propanoate (step 3, 476.9 mg, 1.012 mmol) in tetrahydrofuran (8 mL) and ethanol (8 mL) was added 2 N sodium hydroxide aqueous solution (8 mL) at room temperature. The reaction mixture was stirred at 50° C. for 7 hours and then allowed to warm to room temperature and concentrated in vacuo. The residual solid was dissolved in water (8 mL)- tetrahydrofuran (8 mL), adjusted to pH 4 by adding 2N HCl, then, the mixture was extracted with ethyl acetate (30 mL×3). The combined extracts were dried over magnesium sulfate, and concentrated in vacuo. The residue was purified by preparative TLC on silica gel, developing with CH₂Cl₂/MeOH (15/1), to afford 438.6 mg (97.6%) of the title compound as a white solid:

¹H-NMR (600 MHz, DMSO-d₆, ppm) δ 7.44-7.39 (2H, m), 7.30-7.24 (2H, m), 7.05-6.88 (3H, m), 3.77 (2H, t, J=5.5 Hz), 3.43 (2H, m), 3.12 (1H, dd, J=14 Hz, 6.7 Hz), 2.91-2.60 (6H, m), 2.08-1.97 (6H, m), 1.83-1.72 (2H, m).

MS (ESI positive) m/z: 444 (M+H)⁺, MS (ESI negative) m/z: 442 (M−H)⁻.

IR(KBr): 3427, 2956, 2944, 2860, 1590, 1498, 1473, 1374, 1092, 857 cm⁻¹.

Anal. Calcd for C25H27NO3FCl-1.2H2O: C, 64.50; H. 6.37; N. 3.01.

Found: C, 64.27; H. 5.97; N. 3.04.

### Example 8

### 2-(2-CHLOROBENZYL)-3-(6′-FLUORO-3′H,8H-SPIRO[8-AZABICYCLO[3.2.1]OCTANE-3,1′-[2]BENZOFURAN]1-8-YL)PROPANOIC ACID

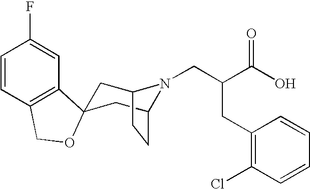

### STEP 1. Ethyl 2-(2-chlorobenzyl)-3-(6′-fluoro-3′H,8H-spiro[8-azabicyclo[3.2.1]octane -3,1′-[2]benzofuran]-8-yl)propanoate

According to the procedure described in step 3 of example 7, 291.5 mg of the title compound was prepared in 36.4% yield from 6′-fluoro-3′H-spiro[8-azabicyclo[3.2.1]octane-3,1′-[2]benzofuran] (408.1 mg, 1.75 mmol) (step 4 of example 3), and ethyl 2-(2-chlorobenzyl)acrylate (453.1 mg, 2.02 mmol) (step 2 of example 7):

¹H-NMR (300 MHz, CDCl₃, ppm) δ 7.39-7.33 (1H, m), 7.26-7.13 (3H, m), 7.08 (1H, dd, J=8.1 Hz, 5.1 Hz), 6.90 (1H, ddd, J=8.1 Hz, 8.1 Hz, 2.2 Hz), 6.68 (1H, dd, J=8.8 Hz, 2.2 Hz), 4.94 (2H, s), 4.10 (2H, q, J=7.3 Hz), 3.28-3.14 (3H, m), 3.02-2.54 (4H, m), 2.19-1.77 (8H, m), 1.18 (3H, t, J=7.3 Hz)l

MS (ESI positive) m/z: 458 (M+H)⁺.

### STEP 2. 2-(2-Chlorobenzyl)-3-(6′-fluoro-3′H,8H-spiro[8-azabicyclo[3.2.1]octane-3,1′-[2]benzofruan]-8-yl)propanoic acid

According to the procedure described in step 4 of example 7, 122.2 mg of the title compound was prepared in 56.8% yield from ethyl 2-(2-chlorobenzyl)-3-(6′-fluoro-3′H,8H-spiro[8-azabicyclo[3.2.1]octane-3,1′-[2]benzofuran]-8-yl)propanoate (step 1, 291.5 mg, 0.637 mmol):

¹H-NMR (600 MHz, DMSO-d₆, ppm) δ 7.42 (1H, d, J=7.8 Hz), 7.39 (1H, dd, J=7.3 Hz, 1.2 Hz), 7.29-7.23 (3H, m), 7.07 (1H, ddd, J=9.3 Hz, 9.3 Hz, 2.1 Hz), 6.76 (1H, dd, J=8.7 Hz, 2.1 Hz), 4.91 (2H, s), 3.36 (2H, m), 3.05-2.95 (2H, m), 2.84-2.73 (2H, m), 2.61 (1H, dd, J=12.1 Hz, 5.7 Hz), 2.12 (2H, m), 2.01-1.75 (6H, m);

MS (ESI positive) m/z: 430 (M+H)⁺, MS (ESI negative) m/z: 428 (M−H)⁻.

IR(KBr): 3400, 3056, 2958, 2915, 2841, 1620, 1480, 1389, 1034, 818, 775 cm ⁻¹.

Anal. Calcd for C24H25NO3FCl-0.4H2O: C, 65.94; H. 5.95; N. 3.20.

Found: C, 65.98; H. 5.80; N. 3.23.

### Example 9

### 2-(2-CHLORO-5-HYDROXYBENZYL)-3-(6′-FLUORO-3′,4′-DIHYDRO-8H-SPIRO[8-AZABICYCLO[3.2.1]OCTANE-3,1′-ISOCHROMEN]-8-YL)PROPANOIC ACID

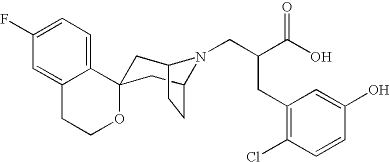

### STEP 1. Ethyl 3-(5-{[tert-butyl(dimethyl)silyl]oxy}-2-chlorophenyl)-2-(diethoxyphosphoryl)propanoate

To a stirred solution of ethyl (diethoxyphosphoryl)acetate (7.062 g, 31.5 mmol) in N,N-dimethylformamide (50.4 mL) was added 60% sodium hydride in mineral oil (1.26 g, 31.5 mmol) at 0° C. and the mixture was stirred at the same temperature for 1.5 hours. To the resulting red solution was added dropwise a solution of [3-(bromomethyl)-4-chlorophenoxy](tert-butyl)dimethylsilane (J. Org. Chem. 1996, 61, 6974.) (10.072 g, 30.0 mmol) in N,N-dimethylformamide (12 mL) at 0° C over a period of 15 minutes, and the resulting mixture was stirred for 4 days at the room temperature. The reaction mixture was poured into water (200 mL) and then extracted with ethyl acetate (150 mL×2). The combined extracts were dried over magnesium sulfate, and concentrated in vacuo. The residue was purified by column chromatography on silica gel, eluting with hexane/ethyl acetate (2/1), to afford 8.3392 g (58%) of the title compound as light brown oil:

¹H-NMR (300 MHz, CDCl₃, ppm) δ 7.17 (1H, d, J=8.8 Hz), 6.76 (1H, d, J=2.9 Hz), 6.65 (1H, dd, J=8.8 Hz, 2.9 Hz), 4.2 (6H, m), 3.47-3.14 (3H, m), 1.39-1.33 (6H, m), 1.19 (3H, t, J=7.34 Hz), 0.94 (9H, s), 0.17 (6H, s); MS (ESI positive) m/z: 479 (M+H)⁺.

### STEP 2. Ethyl 2-(5-{[tert-butyl(dimethyl)silyl]oxy}-2-chlorobenzyl)acrylate

To a stirred mixture of ethyl 3-(5-{[tert-butyl(dimethyl)silyl]oxy}-2-chlorophenyl)-2-(diethoxyphosphoryl)propanoate (step 1, 8.3392 g, 17.4 mmol) and 37% formaldehyde in water (8 mL) was added a solution of potassium carbonate (7.215 g, 52.2 mmol) in water (33.3 mL) at room temperature and the mixture was stirred for 15 hours under reflux. After cooling to room temperature, the reaction mixture was poured into ethyl acetate (100 mL), washed with water (60 mL), dried over magnesium sulfate, and concentrated in vacuo. The residue was purified by column chromatography on silica gel, eluting with hexane/ethyl acetate (12/1), to afford 2.2172 g (35.9%) of the title compound as a colorless oil:

¹H-NMR (270 MHz, CDCl₃, ppm) δ 7.20 (1H, d, J=8.6 Hz), 6.72-6.65 (2H, m), 6.27 (1H, s), 5.34 (1H, d, J=1.3 Hz), 4.22 (2H, q, J=7.3 Hz), 3.68 (2H, s), 1.29 (3H, t, J=7.3 Hz), 0.96 (9H, s), 0.17 (6H, s).

### STEP 3. Ethyl 2-(5-{[tert-Butyl(dimethyl)silyl]oxy}-2-chlorobenzyl)-3-(6′-fluoro-3′, 4′-dihydro-8H-spiro[8-azabicyclo[3.2.1]octane-3,1′-isochromen]-8-yl)propanoate

According to the procedure described in step 3 of example 7, 437.4 mg of the title compound was prepared in 41.3% yield from 6′-fluoro-3′,4′-dihydrospiro[8-azabicyclo[3.2.1]octane-3,1′-isochromene] (step 4 of example 6, 524.3 mg, 2.12 mmol) and ethyl 2-(5-{[tert-butyl(dimethyl)silyl]oxy}-2-chlorobenzyl)acrylate (step 2, 626.2 mg, 1.76 mmol):

¹H-NMR (300 MHz, CDCl₃, ppm) δ 7.19 (1H, d, J=8.8 Hz), 7.65 (1H, dd, J=8.8 Hz, 5.6 Hz), 6.84 (1H, ddd, J=8.8 Hz, 8.8 Hz, 2.9 Hz), 6.75-6.62 (3H, m), 4.12 (2H, q, J=7.3 Hz), 3.81 (2H, t, J=5.1 Hz), 3.25-3.12 (3H, m), 2.99-2.50 (6H, m), 2.11-1.76 (8H, m), 1.21 (3H, t, J=7.3 Hz), 0.97 (9H, s), 0.18 (6H, s);

MS (ESI positive) m/z: 602 (M+H)⁺.

### STEP 4. 2-(2-Chloro-5-hydroxybenzyl)-3-(6′-fluoro-3′,4′-dihydro-8H-spiro[8-azabicyclo[3.2.1]octane-3,1′-isochromen]-8-yl)propanoic acid

To a stirred solution of ethyl 2-(5-{[tert-butyl(dimethyl)silyl]oxy}-2-chlorobenzyl)-3-(6′-fluoro-3′,4′-dihydro-8H-spiro[8-azabicyclo[3.2.1]octane-3,1′-isochromen]-8-yl)propanoate (step 3, 437.4 mg, 0.726 mmol) in tetrahydrofuran (4 mL) and ethanol (4 mL) was added 2 N sodium hydroxide aqueous solution (4 mL) at room temperature. The reaction mixture was stirred at 50° C. for 10 hours and then allowed to warm to room temperature and concentrated in vacuo. The residual solid was dissolved in water (5 mL)-tetrahydrofuran (3 mL)- ethanol (3 mL), adjusted to pH 4 by adding 2N HCl, then, the mixture was extracted with ethyl acetate (30 mL×4). The combined extracts were dried over magnesium sulfate, and concentrated in vacuo. The residue was dissolved in MeOH, and purified by preparative-TLC on silica gel, developing with CH₂Cl₂/MeOH (14/1×1, 12/1×1, and 10/1×2, successively), to afford 40.3 mg of the title compound as a white solid. Then, 22 mg of the solid was dissolved in 25% anmonia-DMSO-MeOH, and purified by HPLC (Waters FractionLynx UV auto-purification system; 254 nm; column: Waters XTerra MS C18, 5 μm, 20×50 mm; eluent: CH₃CN/0.01% aqueous ammonia =20/80 to 40/60 (Gradient); room temperature; flow rate: 20 mL/min) to give 7.0 mg of the title compound as a white solid.

¹H-NMR (600 MHz, DMSO-d₆, ppm) δ 9.61 (1H, brs), 7.19 (1H, d, J=8.6 Hz), 7.04 (1H, ddd, J=8.6 Hz, 8.6 Hz, 2.6 Hz), 6.97-6.95 (1H, m), 6.90 (1H, dd, J=9.6 Hz, 2.5 Hz), 6.79 (1H, d, J=2.8 Hz), 6.65 (1H, dd, J=8.6 Hz, 2.8 Hz), 3.79 (2H, t, J=5.4 Hz), 3.42 (2H, m), 3.01-2.63 (7H, m), 2.07-1.74 (8H, m);

MS (ESI positive) m/z: 460 (M+H)⁺, MS (ESI negative) m/z: 458 (M−H)⁻.

IR(KBr): 3520, 2940, 2590, 1592, 1569, 1475, 1337, 1244, 1108, 1089, 992, 860, 816, 668, 637 cm⁻¹.

### Example 10

### 2-(2-CHLORO-5-HYDROXYBENZYL)-3-(6′-FLUORO-3′H,8H-SPIRO[8-AZABICYCLO[3.2.1]OCTANE-3,1′-[2]BENZOFURAN]-8-YL)PROPANOIC ACID

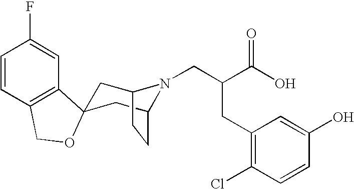

### STEP 1

### Ethyl 2-(5-{[tert-butyl(dimethyl)silyl]oxy}-2-chlorobenzyl)-3-(6′-fluoro-3′H,8H,-spiro[8-azabicyclo[3.2.1]octane-3,1′-[2]benzofuran]-8-yl)propanoate

According to the procedure described in step 4 of example 9, 114.0 mg of the title compound was prepared in 56.8% yield from 6′-fluoro-3′H-spiro[8-azabicyclo[3.2.1]octane-3,1′-[2]benzofuran] (step 4 of example 3, 84.3 mmol, 0.36 mmol) and ethyl 2-(5-{[tert-butyl(dimethyl)silyl]oxy}-2-chlorobenzyl)acrylate (step 2 of example 9 147.9 mg, 0.42 mmol):

¹H-NMR (300 MHz, CDCl₃, ppm) δ 7.18 (1H, d, J=8.8 Hz), 7.08 (1H, dd, J=8.1 Hz, 5.1 Hz), 6.88 (1H, ddd, J=8.8 Hz, 8.8 Hz, 2.2 Hz), 6.73-6.63 (3H, m), 4.94 (2H, s), 4.12 (2H, m), 3.24 (2H, brs), 3.11 (1H, dd, J=12.5 Hz, 4.4 Hz), 2.99-2.52 (4H, m), 2.19-1.76 (8H, m), 1.22 (3H, t, J=7.3 Hz), 0.96 (9H, s), 0.18 (6H, s); MS (ESI positive) m/z: 588 (M+H)⁺.

### STEP 2

### 2-(2-Chloro-5-hydrobenzyl)-4-(6′-fluro-3′H,8H-spiro[8-azabicyclo[3.2.1]octane-3,1′-[2]-benzofuran]-8-yl)propanoic acid

According to the procedure described in step 4 of example 9, 1.1 mg of the title compound was prepared from ethyl 2-(5-{[tert-butyl(dimethyl)silyl]oxy}-2-chlorobenzyl)-3-(6′-fluoro-3′H,8H-spiro[8-azabicyclo[3.2.1]octane-3,1′-[2]benzofuran]-8-yl)propanoate (step 1, 114.0 mg, 0.194 mmol).

¹H-NMR (600 MHz, DMSO-d₆, ppm) δ 9.64 (1H, brs), 7.27 (1H, dd, J=8.3 Hz, 5.0 Hz), 7.17 (1H, d, J=8.6 Hz), 7.07 (1H, ddd, J=8.4 Hz, 8.4 Hz, 2.3 Hz), 6.79-6.76 (2H, m), 6.62 (1H, dd, J=8.6 Hz, 2.9 Hz), 4.91 (2H, s), 3.33 (2H, -m), 2.89 (2H, d, J=6.3 Hz), 2.76-2.57 (3H, m), 2.14-1.75 (8H, m);

MS (ESI positive) m/z: 446 (M+H)⁺, MS (ESI negative) m/z: 444 (M−H)⁻.

### Example 11

### SODIUM 2-(2-CHLOROBENZYL)-3-(6′-FLUORO-3′,4′-DIHYDRO-8H-SPIRO[8-AZABICYCLO[3.2.1]OCTANE-3,1′-ISOCHROMEN]-8-YL)PROPANOATE

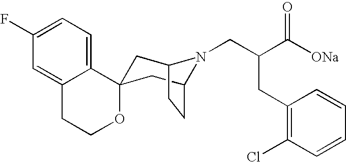

To a stirred suspension of 2-(2-Chlorobenzyl)-3-(6′-fluoro-3′,4′-dihydro-8H-spiro[8-azabicyclo[3.2.1]octane-3,1′-isochromen]-8-yl)propanoic acid (step 4 of example 7, 285 mg, 0.642 mmol and 0.1 N NaOH aqueous solution (6.4 ml, 0.64 mmol) was added ethanol (2 ml) dropwise at room temperature. The reaction mixture turned to a clear solution. After 30 minutes stirring, the reaction mixture was concentrated and dried under vacuum at room temperature to afford 315 mg of the title compound as white solid.

Anal.Calcd.for C₂₅H₂₆NO₃FCINa-2.5 H₂O: C, 58.77; H, 6.12; N, 2.74.

Found: C, 58.46; H, 5.87; N, 2.64.

### Example 12

### SODIUM 2-(2-CHLOROBENZYL)-3-(6′-FLUORO-3′H,8H-SPIRO[8-AZABICYCLO[3.2.1]OCTANE-3,1′-[2]BENZOFURAN]1-8-YL)PROPANOATE

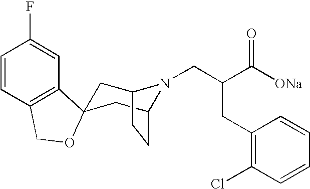

To a stirred suspension of 2-(2-Chlorobenzyl)-3-(6′-fluoro-3′H,8H-spiro[8-azabicyclo[3.2.1]octane-3,1′-[2]benzofuran]-8-yl) propanoic acid (step 2 of example 8, 111 mg, 0.258 mmol) and 0.1 N NaOH aqueous solution (2.58 ml, 0.258 mmol) was added ethanol (2 ml) dropwise at room temperature. The reaction mixture turned to a clear solution. Then the reaction mixture was concentrated and dried under vacuum at room temperature to afford 117 mg of the title compound as a white solid.

Anal.Calcd.for C₂₄H₂₄NO₃FCINa-3.5 H₂O: C, 55.98; H, 6.07; N, 2.72.

Found: C, 55.68; H, 5.73; N, 2.60.

1-13. (canceled)

14. A compound of the formula (I) 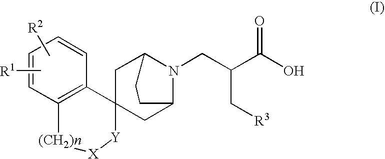
wherein R¹ and R² are independently selected from hydrogen, halogen, or (C₁-C₃)alkyl,
R³ is aryl or heteroar, each optionally substituted by 1 to 3 substituents independently selected from halogen, hydroxy, (C₁-C₃)alkyl, or (C₁-C₃)alkoxy, wherein said heteroaryl is a 5- or 6-membered aromatic heterocyclic group comprising (a) 1 to 4 nitrogens, (b) 1 oxygen or 1 sulphur: or (c) 1 oxygen or 1 sulphur and 1 or 2 nitrogens;
—X—Y— is —CH₂O—, —CH(CH₃)O—, or —C(CH₃)₂O—;
and n is 0, 1, or 2;
or a pharmaceutically acceptable ester or salt thereof.

15. The compound of claim 14: wherein R¹ and R² are independently hydrogen or fluorine, or a pharmaceutically acceptable salt thereof.

16. The compound of claim 14, wherein p³ is phenyl or heteroaryl, each optionally substituted by 1 to 3 substituents independently selected from halogen, hydroxy, (C₁-C₃)alkyl, or (C₁-C₃)alkoxy, wherein said heteroaryl is a 5- or 6-membered aromatic heterocyclic comprising either (a) 1 to 2 nitrogens, or (b) 1 oxygen or 1 sulphur and 1 or 2 nitrogens, or a pharmaceutically acceptable salt thereof.

17. The compound of claim 15, wherein R³ is phenyl or heteroaryl, each optionally substituted by 1 to 3 substituents independently selected from halogen, hydroxy, (C₁-C₃)alkyl, or (C₁-C₃)alkoxy, wherein said heteroaryl is a 5- or 6-membered aromatic heterocyclic comprising either (a) 1 to 2 nitrogens, or (b) 1 oxygen or 1 sulphur and 1 or 2 nitrogens, or a pharmaceutically acceptable salt thereof.

18. The compound of claim 14, wherein R³ is phenyl or heteroaryl selected from pyridyl, thiazolyl, isothiazolyl, pyrazolyl, imidazolyl, isoxazolyl or oxazolyl; said phenyl and heteroaryl optionally substituted by 1 to 2 substituents each independently selected from halogen, hydroxy, or methyl, or a pharmaceutically acceptable salt thereof.

19. The compound of claim 15, wherein R³ is phenyl or heteroaryl selected from pyridyl, thiazolyl, isothiazolyl, pyrazolyl, imidazolyl, isoxazolyl or oxazolyl; said phenyl and heteroaryl optionally substituted by 1 to 2 substituents each independently selected from halogen, hydroxy, or methyl, or a pharmaceutically acceptable salt thereof.

20. The compound of claim 14, wherein R³ is phenyl or heteroaryl selected from thiazolyl or pyrazolyl, said phenyl and heteroaryl are optionally substituted by 1 to 2 substituents each independently selected from halogen or hydroxy, or a pharmaceutically acceptable salt thereof.

21. The compound of claim 15, wherein R³is phenyl or heteroaryl selected from thiazolyl or pyrazolyl, said phenyl and heteroaryl are optionally substituted by 1 to 2 substituents each independently selected from halogen or hydroxy, or a pharmaceutically acceptable salt thereof.

22. The compound of claim 14, wherein —X—Y— is —CH₂O—, or a pharmaceutically acceptable salt thereof.

23. The compound of claim 21, wherein —X—Y— is —CH₂O—, or a pharmaceutically acceptable salt thereof.

24. The compound claim 14, wherein n is 0 or 1, or a pharmaceutically acceptable salt thereof.

25. The compound claim 21, wherein n is 0 or 1, or a pharmaceutically acceptable salt thereof.

26. The compound claim 23, wherein n is 0 or 1, or a pharmaceutically acceptable salt thereof.

27. The compound of claim 14, which is selected from:
3-(3′H ,8H-Spiro[8-azabicyclo[3.2.1]octane-3,1′-[2]benzofuran]-8-yl)-2-(1,3-thiazol-4-ylmethyl)propanoic acid;
3-(1H-Pyrazol-1-yl)-2-(3′H8H-spiro[8-azabicyclo[1.2.1]octane-3,1′-[2]-benzofuran]-8-ylmethyl)propanoic acid;
6′-fluoro-3′H,8H-spiro[8-azabicyclo[3.2.1]octane-3,1′-[2]benzofuran]-8-carboxylate;
3-(6′-Fluoro-3′H ,8H-spiro[8-azabicyclo[3.2.1]octane-3,1′-[2]benzofuran]-8-yl)-2-(1,3-thiazol-4-ylmethyl)propanoic acid;
3-(3′,4′-Dihydro-8H-spiro[8-azabicyo[3.2.1]octane-3,1′-isochromen]-8-yl)-2-(1H-pyrazol-1-ylmethyl)propanoic acid;
3-(6′-fluoro-3′,4′-dihydro-8H-spiro[8-azabicyclo[3.2.1]ocane-3,1′-isochromen]-8-yl)-2-(1H-pyrazol-1-ylmethyl)propanoic acid;
2-(2-Chlorobenzyl)-3-(6′-fluoro-3′,4′-dihydro-8H-spiro[8-azabicyclo[3.2.1]octane-3,1′isochromen]-8-yl)propanoic acid;
2-(2-Chlorobenzyl)-3-(6′-floro-3′H,8H-spiro[8-azabicyclo[3.2.1]octane-3,1′-[2]benzofuran]-8yl)propanoic acid;
2-(2-Chloro-5-hydroxybenzyl)-3-(6′-fluoro-3′,4′-dihydro-8H-spiro[8-azabicyclo[3.2.1]octane-3,1′-isochromen]-8-yl)propanoic acid;
2-(2-Chloro-5-hydroxybenzyl)-3-(6′-fluoro-3′H,8H-spiro[8-azabicyclo[3.2.1]octane-3,1′-[2]benzofuran]-8-yl)propanoic acid,;
or a pharmaceutically acceptable salt thereof.

28. A pharmaceutical composition comprising the compound of claim 14, or a pharmaceutically acceptable salt thereof, together with at least one pharmaceutically acceptable excipient.

29. A method of treating a disease for which an ORL1 antagonist is indicated, comprising administering an effective amount of the compound of claim 14, or a pharmaceutically acceptable salt thereof, to a subject in need thereof.
